SEQUENCE LISTING (1 sequence)

SEQ ID NO: 1 - DNA, 20 bp, Rattus norvegicus
gctactgccgccgggacatc

(19) United States
(12) Patent Application Publication
(10) Pub. No.: US 2022/0280460 A1
(43) Pub. Date: Sep. 8, 2022

(54) METHODS AND COMPOSITIONS FOR STIMULATING EXOSOME SECRETION

(71) Applicant: UNIVERSITY OF PITTSBURGH-OF THE COMMONWEALTH SYSTEM OF HIGHER EDUCATION, Pittsburgh, PA (US)

(72) Inventors: Edwin Kerry JACKSON, Pittsburgh, PA (US); Theresa L. WHITESIDE, Pittsburgh, PA (US); Nils LUDWIG, Regensburg (DE)

(21) Appl. No.: 17/637,881

(22) PCT Filed: Aug. 7, 2020
(86) PCT No.: PCT/US2020/045339

§ 371 Date: Feb. 24, 2022

Related U.S. Application Data
(60) Provisional application No. 62/892,247, filed on Aug. 27, 2019.

(51) Int. Cl. A61K 31/19; G01N 33/574; C12Q 1/6886; A61K 31/06

(52) U.S. Cl. CPC A61K 31/19; G01N 33/57488; C12Q 1/6886; A61K 31/06

(57) ABSTRACT

Disclosed herein are methods for improving exosome production or stimulating exosome secretion by cells. The methods comprise inhibiting at least one step of the glycolytic pathway by contacting the cells with at least one glycolytic inhibitor and/or inhibiting mitochondrial function by contacting the cells with at least one inhibitor of mitochondrial function.

### STATEMENT REGARDING FEDERALLY SPONSORED RESEARCH OR DEVELOPMENT

This invention was made with Government Support under Grant No. DK091190 awarded by the National Institutes of Health. The Government has certain rights in the invention.

### FIELD

The disclosure generally relates to methods and compositions for improving exosome production or stimulating exosome secretion.

### BACKGROUND

Extracellular vesicles, especially exosomes in the nano-size range of 30-150 nm, have shown important roles in intercellular communications in recent decades. The formation of exosomes begins with the creation of endosomes as the intracellular vesicles. Exosomes are differing from other membrane-derived microvesicles by originating from multivesicular bodies (MVBs) for cellular secretion. Therefore, exosomes contain specific proteins and nucleic acids and represent their parent cell status and functions at the time of formation in parent cells. Compared to other nano-sized delivery systems, such as lipid, polymers, gold and silica material, exosomes are living-cell derived, highly biocompatible nano-carriers with intrinsic payload, and exhibit much stronger flexibility in loading desired antigens for effective delivery. Exosomes also eliminate allergenic responses without concerns of carrying virulent factors and avoid degradation or loss during delivery. However, the development of exosome-based vaccines is hindered by substantial technical difficulties in obtaining pure immunogenic exosomes.

Extracellular vesicles (EVs), including the small subset of EVs referred to as exosomes which are derived from the endocytic compartment of parental cells and range in size from 30 to 150 nm, play an important role in intercellular communication. The biogenesis of exosomes is distinct from that of other EVs, such as microvesicles (MVs) or apoptotic bodies. It begins with the internalization of cell surface proteins by endocytosis and the sequestration of these proteins by early endosomes. In late endosomes, a process of reverse vesicular invagination leads to the formation of multivesicular bodies (MVBs), which are filled with numerous vesicles. Importantly the topography of proteins decorating these vesicles mimics that of the surface membrane of a parental cell. Thus, exosomes differ from other EVs in that their vesicular cargo is derived from the proteins processed in late endosomes and packaged into vesicles in the MVBs. When MVBs fuse with the cell membrane of the parent cell, exosomes are released into the extracellular space. Exosome packaging and their cellular secretion have been investigated and while the general mechanistic underpinning their formation are described, it remains unclear to which extent the packaged and secreted exosomes are molecular mimics of their parental cells or whether they carry addressed instructions to the potential recipient cells. Nevertheless, secreted and circulating exosomes are looked upon as a liquid biopsy, and their characteristics, cargos of proteins and nucleic acids and their identity with the parent cells have been of great interest.

Exosomes are in demand not only as potential non-invasive biomarkers but also as a delivery system of messages that can be transferred to or incorporated into the exosome cargo. Compared to other nano-sized delivery systems, such as lipids, polymers, gold and silica materials, exosomes are living-cell derived, highly biocompatible nano-carriers with an intrinsic payload that can be experimentally modified. Exosomes are characterized by much greater flexibility in loading desired antigens for effective delivery than are, e.g, liposomes. Exosomes are reported to remain in the circulation longer than liposomes, and they interact with a broad variety of cell targets, including dendritic cells (DCs). Exosome cargos avoid degradation or loss during delivery to distant sites. Due to these characteristics, exosomes are considered to be favorable component of vaccines. However, the development of exosome-based vaccines, as well as other applications of exosomes, has been hindered by substantial technical difficulties in obtaining immunogenic exosomes that are free of plasma-derived or cell supernatant-derived “contaminants” in quantities adequate for ex vivo studies.

Although all cells secrete exosomes, there are substantial differences between cells in levels of released exosomes. Specifically, cultured tumor cells secrete enlarged quantities of exosomes, while normal tissue cells secrete small-to-moderate quantities.

There is a need for standardizing and optimizing exosome production, for example, by cultured cell lines. The compositions and methods disclosed herein address these and other needs.

### SUMMARY

In the present disclosure, methods for improving production or stimulating the secretion of exosomes are disclosed. In certain embodiments, the methods can include inhibiting at least one step of the glycolytic pathway by contacting the cells with at least one glycolytic inhibitor and/or inhibiting mitochondrial function by contacting the cells with at least one inhibitor of mitochondrial function. For example, the methods for improving exosome production or stimulating exosome secretion by cells can include inhibiting at least one step of the glycolytic pathway by contacting the cells with at least one glycolytic inhibitor. In other examples, the methods for improving exosome production or stimulating exosome secretion by cells can include inhibiting mitochondrial function by contacting the cells with at least one inhibitor of mitochondrial function. In further examples, the methods for improving exosome production or stimulating exosome secretion by cells can include both steps of inhibiting at least one step of the glycolytic pathway by contacting the cells with at least one glycolytic inhibitor, and inhibiting mitochondrial function by contacting the cells with at least one inhibitor of mitochondrial function. The methods can further include culturing a population of the cells to increase the number of cells prior to inhibiting the glycolytic pathway or mitochondrial function.

Glycolytic inhibitors are known and can include a pharmacological agent that can inhibit any of the enzymes within the glycolysis pathway. For example, glycolytic inhibitors can include a pharmacological agent that can inhibit glucose transporter, hexokinase, phosphofructokinase, glyceraldehyde-3-phosphate dehydrogenase, phosphoglycerate mutase 1, pyruvate kinase M2, lactate dehydrogenase A, monocarboxylate transporters, pyruvate dehydrogenase, phosphoglucose isomerase, aldolase, enolase, phosphoglycerate kinase, or combinations thereof. In some examples, the glycolytic inhibitor can include a pharmacological agent that inhibits glyceraldehyde-3-phosphate dehydrogenase. Specific examples of glycolytic inhibitors include a pharmacological agent selected from a halogenated acetate, a monosaccharide or derivative thereof, valerate or derivative thereof, a propionic acid derivative, a pyruvate derivative, or a combination thereof. Preferably, the glycolytic inhibitor is a halogenated acetate, such as, iodoacetate.

Inhibitors of mitochondrial function are also known and can include any pharmacological agent that can inhibit any of the multiple functions of the mitochondria. Indeed, one role of the mitochondria is in cell metabolism or regulation of bioenergetics pathways. Pharmacological agents that inhibit any of these functions are considered herein. In some embodiments, the inhibitor of mitochondrial function can be a pharmacological agent that inhibits oxidative phosphorylation. For example, the inhibitor of mitochondrial function can include 2,4-dinitrophenol (DNP).

In specific examples, the methods for improving production or stimulating the secretion of exosomes can include contacting the cells with iodoacetate and 2,4-dinitrophenol. The iodoacetate and 2,4-dinitrophenol can be present in a molar ratio from 1:10 to 10:1, preferably from 1:2 to 2:1, more preferably 1:1.

The methods disclosed herein can be carried out in vitro or in vivo. In any of these methods, the cells can be obtained from or present in a subject's bodily fluid, skin, skeletal muscle, brain, heart, gut, liver, ovarian epithelium, umbilical cord, testis, tissue, membranous lining (including the meninges, pericardium, pleura, or peritoneum), or stem cells. In some examples, the cells can be obtained from or present in the subject's bodily fluid such as from cord blood, peripheral blood, brain, blood vessels, serum, plasma, ascites, urine, cerebrospinal fluid (CSF), sputum, saliva, bone marrow, synovial fluid, aqueous humor, cerumen, bronchioalveolar lavage fluid, semen, prostatic fluid, cowper's fluid or pre-ejaculatory fluid, sweat, fecal matter, tears, cyst fluid, pleural and peritoneal fluid, lymph, chyme, chyle, bile, intestinal fluid, pus, sebum, vomit, mucosal secretion, stool water, pancreatic juice, lavage fluids from sinus cavities, or bronchopulmonary aspirates. The cells can be tumor cells or cells affected by other conditions.

In one example disclosed herein, cultured cells were treated or not with sodium iodoacetate (IAA; glycolysis inhibitor) plus 2,4-dinitrophenol (DNP; oxidative phosphorylation inhibitor). Exosomes were isolated by size-exclusion chromatography and their morphology, size, concentration, cargo components and functional activity were compared. IAA/DNP treatment (up to 10 μM each) was non-toxic and resulted in at least 5 fold, at least 10 fold (such as from 3 fold to 16 fold) increase in exosome secretion compared to uninhibited cells. Exosomes from IAA/DNP-treated or untreated cells had similar biological properties and functional effects on endothelial cells (SVEC4-10). IAA/DNP increased exosome secretion from mouse organ cultures, and in vivo injections enhanced the levels of circulating exosomes. IAA/DNP decreased ATP levels (p<0.05) in cells. A cell membrane-permeable form of 2′,3′-cAMP and 3′-AMP mimicked the potentiating effects of IAA/DNP on exosome secretion. In cells lacking 2′,3′-cyclic nucleotide 3′-phosphodiesterase (CNPase; an enzyme that metabolizes 2′,3′-cAMP into 2′-AMP), effects of IAA/DNP on exosome secretion were enhanced. The IAA/DNP combination is a powerful stimulator of exosome secretion, and these stimulatory effects are, in part, mediated by intracellular 2′,3′-cAMP.

Compositions comprising exosomes, wherein the exosomes are prepared by a method as disclosed herein are also disclosed. The compositions comprise exosomes in an amount of at least 5 fold, at least 10 fold, or at least 15 fold, compared to uninhibited cells. The compositions may further comprise at least one glycolytic inhibitor and/or at least one inhibitor of mitochondrial function.

Methods for characterizing a condition using the methods for improving production or stimulating the secretion of exosomes are also disclosed. The methods for characterizing a condition can comprise stimulating cells in a sample to produce exosomes comprising inhibiting at least one step of the glycolytic pathway by contacting the cells with at least one glycolytic inhibitor, and/or inhibiting mitochondrial function by contacting the cells with at least one inhibitor of mitochondrial function; determining or identifying a biosignature of the isolated exosomes; and characterizing the condition based on the biosignature. The methods for characterizing a condition can be carried out in vitro or in vivo. The in vivo methods for characterizing a condition can include stimulating cells in a subject to produce exosomes comprising inhibiting at least one step of the glycolytic pathway by contacting the cells with at least one glycolytic inhibitor, and/or inhibiting mitochondrial function by contacting the cells with at least one inhibitor of mitochondrial function; collecting a sample comprising the exosomes from the subject; determining or identifying a biosignature of the exosomes; and characterizing the condition based on the biosignature. The cells can be obtained from or present in a tissue, a stem cell, or a membranous lining. In some examples, the cells are obtained from or present in a membranous lining in the thoracic cavity, cranial cavity, abdominal cavity, spinal cavity, pelvic cavity, oral cavity, nasal cavity, orbital cavity, or synovial cavity.

The methods for characterizing a condition can further include isolating the exosomes prior to the step of determining or identifying a biosignature of the exosomes. The exosomes can be isolated using size exclusion chromatography, density gradient centrifugation, differential centrifugation, nanomembrane filtration (e.g., ultrafiltration or microfiltration), tangential flow filtration, hydrostatic filtration dialysis, immunoisolation, affinity purification, microfluidic separation, precipitation, field-flow fractionation, asymmetric flow field-flow fractionation, field-free viscoelastic flow, electrophoretic, acoustic, ion exchange chromatography, fast protein/high performance liquid chromatography (FPLC/HPLC), fluorescence-activated sorting, deterministic lateral displacement (DLD) arrays, or a combination thereof.

The biosignature can comprise a polypeptide, protein, lipid, RNA, DNA, antigen, antibody, antibody fragment, aptamer, peptoid, zDNA, peptide nucleic acid (PNA), locked nucleic acids (LNA), purines, metabolites, or modifications thereof, that are associated with the condition. Determining or identifying a biosignature of the exosomes can comprise measuring an expression level, presence, absence, mutation, truncation, insertion, modification, sequence variation or molecular association of the biosignature from the isolated exosome. Characterizing the condition can include comparing the biosignature to a reference followed by a diagnosis, prognosis, determination of drug efficacy, monitoring the status of the cell's response or resistance to a treatment, or selection of a treatment for the condition. In some cases, an elevated presence or level of the biosignature as compared to the reference indicates that the cells are predisposed to or afflicted with the condition. The condition can be cancer or diseases of the heart, blood vessels, kidneys, liver, lungs, gastrointestinal tract (such as the colon), bladder, or brain. After the condition has been characterized, the methods disclosed herein can further include administering a therapeutic agent, such as a chemotherapy agent to treat the condition.

Methods for treating a condition in a subject using the methods for improving production or stimulating the secretion of exosomes are also disclosed. The method for treating a condition in the subject can comprise administering to the subject a composition comprising exosomes prepared by a method disclosed herein. The condition can be a disease such as cancer, cardiovascular disease, or disease of an organ system. The exosomes used in the methods can be derived from an autologous source or an allogeneic source. The exosomes can comprise an imaging agent, a therapeutic agent, a detectable moiety, or a combination thereof, preferably a therapeutic agent. For example, the exosomes can comprise a payload selected from peptide, protein, DNA, RNA, siRNA, miRNA, shRNA, small molecule, large molecule biologic, polysaccharide, lipid, toxin or combinations thereof.

### DESCRIPTION OF DRAWINGS

FIG. 1 shows exosome production in response to IAA/DNP. Levels of total exosomal protein in μg normalized to 10⁶ cells derived from UMSCC47 (FIG. 1A), PCI-13 (FIG. 1B) or Mel526 (FIG. 1C) cell lines cultured in absence or presence (1 or 10 μM) of IAA/DNP. Results were validated by qNano for UMSCC47 (FIG. 1D), PCI-13 (FIG. 1E) and Mel526 (FIG. 1F) and expressed as particle concentrations normalized to 10⁶ cells. Values represent means±SEM; *p<0.05; **p<0.01.

FIG. 2 shows the properties of exosomes derived from cells treated with IAA/DNP. (FIG. 2A) TEM images of isolated and negatively-stained UMSCC47-, PCI-13- and Mel526-derived exosomes. Cells were treated with indicated concentrations of IAA/DNP. (FIGS. 2A and 2B) Size distributions of UMSCC47-, PCI-13- and Mel526-derived exosomes were measured by qNano. Values represent means±SD. (FIG. 2C) Western blots of isolated UMSCC47- and PCI-13- and Mel526-derived exosomes with a TSG101 antibody.

FIG. 3 shows the functional activity of exosomes derived from cells treated with IAA/DNP. (FIG. 3A) Levels of total exosomal protein in μg normalized to 10⁶ cells derived from SVEC4-10 cells. (FIG. 3B) Representative images of SVEC4-10 cells treated with indicated concentrations of IAA/DNP. (FIG. 3C) Internalization of UMSCC47-derived exosomes by SVEC4-10 cells after 4 hours. Exosomes were derived from cells treated with the indicated concentrations of IAA/DNP. (FIG. 3D) Migration of SVEC4-10 cells towards serum-free media (Neg. CTRL), 10% FBS (Pos. CTRL) and 10 μg of protein of exosomes derived from cells with the indicated concentrations of IAA/DNP. (FIG. 3E) Representative images of migrated cells in 20× magnification. All values represent means±SEM; *p<0.05.

FIG. 4 shows IAA/DNP stimulates exosome secretion ex vivo and increases circulating levels of exosomes in vivo. (FIGS. 4A, 4B, and 4C) Exosome production by tissue explants in response to IAA/DNP. Harvested kidneys were cultured for 48 hours with the indicated concentration of IAA/DNP. The tissues were cultured intact (FIG. 4A), minced (FIG. 4B) or the treatment was injected into the intact tissue with a syringe (FIG. 4C). Total protein concentrations are expressed in μg and were normalized to 100 mg of tissue. (FIG. 4D) Western blots of isolated exosomes derived from tissue explants with a TSG101 antibody. (FIG. 4E) Plasma levels of total exosomal protein in μg normalized to 100 μl of plasma after 0, 7 and 14 days of treatment. Mice were either treated with PBS, 0.195 μmoles IAA/DNP or 0.975 μmoles of IAA/DNP. Dotted line indicates basal level of circulating exosomes. (FIG. 4F) Body weight (g) of mice after 14 days of treatment. (FIG. 4G) Levels of total exosomal protein in μg normalized to 100 mg of tissue derived from kidneys which were harvested after 14 days of treatment with indicated concentrations of IAA/DNP. (FIG. 4H) Levels of total exosomal protein in μg normalized to 100 mg of tissue derived from livers which were harvested after 14 days of treatment with indicated concentrations of IAA/DNP. All values represent means±SEM; *p<0.05; **p<0.01.

FIG. 5 shows IAA/DNP causes energy depletion in cultured cells. Levels of ATP (FIG. 5A), ADP (FIG. 5B) and AMP (FIG. 5C) were quantitated by HPLC, and the data were corrected for cell number. (FIG. 5D) Based on the data shown in A-C the cellular energy charge was calculated using the indicated formula. (FIG. 5E) Exosome production in response to IAA/DNP in combination with dorsomorphin dihydrochloride. Levels of total exosomal protein in μg normalized to 10⁶ cells derived from UMSCC47 cells. (FIG. 5F) Exosome production in response to IAA/DNP in combination with MRS 1754. Levels of total exosomal protein in μg normalized to 10⁶ cells derived from UMSCC47 cells. Values represent means±SEM; *p<0.05 vs. untreated; **p<0.01 vs. untreated; ***p<0.001 vs. untreated; #p<0.05 vs. IAA/DNP.

FIG. 6 shows exosome production by UMSCC47 cells in response to 8-Br-2′,3′;-cAMP (FIG. 6A), 2,′3′-cAMP (FIG. 6B), 2′-AMP (FIG. 6C) and 3′-AMP (FIG. 6D). Levels of total exosomal protein in μg normalized to 10⁶ cells. (FIG. 6E) Exosome production by PGVSMCs isolated from CNPASE+1+ and −/− rats in response to 8-Br-2′,3′-cAMP (0.3 μM) and IAA/DNP (5 μM). Values represent means±SEM; *p<0.05; **p<0.01.

FIG. 7 shows schematic summarizes the biochemical steps in the stimulation of exosome release by IAA/DNP. The simultaneous inhibition of glycolysis and oxidative phosphorylation leads to energy depletion in the cells (decreased ATP levels, elevated AMP levels). As a result of the energy depletion, the cells release adenosine, which activates the A_{2B} receptor system which then enhances exosome release. Simultaneously, the cells release 2′,3′-cAMP, which stimulates the release of exosomes directly, but can also be a source for adenosine, which again can activate the adenosine receptor system.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Unless otherwise expressly stated, it is in no way intended that any method set forth herein be construed as requiring that its steps be performed in a specific order. Thus, where a method claim does not expressly recite an order of steps to be followed or it is not otherwise specifically stated in the claims or description that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “can,” “may,” “optionally,” “can optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The terms “secretion/secrete/secreting” refer to any release and/or transport of a biological molecule or signal within a cell or from the cytoplasm of a cell across the cytoplasmic membrane. In some examples, the terms “secretion/secrete/secreting” include any release of an exosome from a biological cell into the surrounding medium. Such secretion may be the result of active transport, passive diffusion or cell lysis.

The term “subject” refers to any individual who is the target of administration or treatment. The subject can be a vertebrate, for example, a mammal Thus, the subject can be a human or veterinary patient. The term “patient” refers to a subject that is under the care of a treating clinician (e.g., physician).

Methods of Secreting Extracellular Vesicles

A growing body of interest emphasizes the important roles of extracellular vesicles (from 30 nm-10 μm), particularly exosomes (from 30-150 nm), in different physiological and pathophysiological conditions. For example, exosomes are the emerging cargo for mediating cellular signal transductions. Exosomes can carry numerous cargos, including lipids, proteins, nucleic acids, and metabolites. Exosomal cargos are dependent on the parent cell type and vary between different physiological or pathological conditions in which the donor cells live. Exosomes are also being studied as biomarkers in different diseases and their use as drug carriers in nanomedicine is considered herein. Standard methods for culturing and isolating exosomes lack the ability to isolate exosomes in high yields. The present disclosure addresses needs in the art by providing methods for improving production or stimulating secretion of extracellular vesicle, particularly of exosomes in vitro and in vivo that is applicable to all cell types.

The terms “extracellular vesicle” and “exosomes” are used interchangeably and refer to a class of vesicles formed by inward budding of endosomal membrane and releasing into the extracellular environment upon fusion with the plasma membrane. Exosomes are usually cup-shaped under transmission electron microscopy, and carry specific markers, such as CD63, CD9, CD81, Alix, HSP90 and fibronectin. The size of exosomes can vary from about 20 nm to about 150 nm, such as from 20 nm to 100 nm, from 30 nm to 100 nm, from 40 nm to 100 nm, from 30 nm to 150 nm, from 40 nm to 150 nm, or from 50 nm to 100 nm.

In some aspects, methods for improving exosome production or stimulating exosome secretion in cells comprising inhibiting at least one step of the glycolytic pathway by contacting the cells with at least one glycolytic inhibitor are disclosed. The glycolytic pathway is associated with a number of glycolytic enzymes. The enzymes that are considered targets along the glycolytic pathway include, but are not limited to, glucose transporters, hexokinase, phosphofructokinase, glyceraldehyde-3-phosphate dehydrogenase, phosphoglycerate mutase 1, pyruvate kinase M2, lactate dehydrogenase A, monocarboxylate transporters, pyruvate dehydrogenase, phosphoglucose isomerase, aldolase, enolase, and phosphoglycerate kinase. Inhibition of one or more glycolytic enzymes in the glycolytic pathway has been shown to be effective for improving production or stimulating the secretion of extracellular vesicles. Inhibitors of the glycolytic pathway, particularly, enzymes in the glycolytic pathway can be selected from a small molecule, a peptide, a protein, a DNA, a RNA, a siRNA, a miRNA, a shRNA, a large molecule biologic, a polysaccharide, a toxin, or a combination thereof.

In some embodiments, the methods for improving exosome production or stimulating exosome secretion by cells include inhibiting glyceraldehyde-3-phosphate dehydrogenase by contacting the cells with at least one glyceraldehyde-3-phosphate dehydrogenase inhibitor. Glyceraldehyde-3-phosphate dehydrogenase (GAPDH) is a glycolytic enzyme catalyzing the formation of 1,3-diphosphoglycerate from glyceraldehyde-3-phosphate and inorganic phosphate. GAPDH inhibitors include compounds with high affinity to the NAD-binding site and theoretically capable of forming a disulfide bond with amino acid residue Cys149.

In some embodiments, the methods for improving exosome production or stimulating exosome secretion by cells include inhibiting glucose transporters (GLUT1 to GLUTS) by contacting the cells with at least one inhibitor of glucose transporter. The human GLUT family includes 14 members (GLUT1-14). WZB117 (3-fluoro-1,2-phenylene bis(3-hydroxybenzoate), 3-hydroxy-benzoic acid 1,1′-(3-fluoro-1,2-phenylene) ester) is an inhibitor of GLUT1 that decreases glucose uptake, intracellular ATP levels, and glycolytic enzymes leading to a lowered rate of glycolysis and cellular growth. Other inhibitors of GLUT1 include nitrogen-containing bicyclic heterocycles, benzamide derivatives, quinazoline derivatives, N-pyrazolyl quinoline carboxamides, purinone derivatives, or combinations thereof. Sheng and Tang, Recent Patents on Anti-Cancer Drug Discovery, 2016, 11:297-308 describes several glycolytic inhibitors, and is incorporated herein by reference.

In some embodiments, the methods for improving exosome production or stimulating exosome secretion by cells include inhibiting hexokinase by contacting the cells with at least one hexokinase inhibitor. Hexokinase is the rate-limiting enzyme for the generation of glucose 6-phosphate. Hexokinase inhibitors include 2-deoxy-D-glucose, 3-bromopyruvate, lonidamine, glucosamine and glucosamine derivatives, fluorinated hexopyanose, or combinations thereof.

In some embodiments, the methods for improving exosome production or stimulating exosome secretion by cells include inhibiting phosphofructokinase by contacting the cells with at least one phosphofructokinase inhibitor. Phosphofructokinase (PFK) catalyzes phosphorylation of fructose-6-phosphate to fructose-1,6-bisphosphate. Inhibitors of phosphofructokinase include pyridynyl trifluoromethyl quinolinyl propanone, benzopyran and naphthalene derivatives, heteroaryl sulfonamides, biaryl sulfonamides, or combinations thereof.

In some embodiments, the methods for improving exosome production or stimulating exosome secretion by cells include inhibiting pyruvate kinase by contacting the cells with at least one pyruvate kinase inhibitor. Pyruvate kinase M2 (PKM2) catalyzes the formation of pyruvate and ATP by irreversible phosphoryl group transfer from phosphoenolpyruvate to ADP. Inhibitors of PKM2 include antibodies that inhibit the acetylation of PKM2, vitamin K-like compounds such as vitamin K3 and vitamin K5, natural products such as L-alkannin or D-alkannin, or combinations thereof.

In some embodiments, the methods for improving exosome production or stimulating exosome secretion by cells include inhibiting pyruvate dehydrogenase (PDH). Inhibitors of pyruvate dehydrogenase includes dichloroacetate (DCA), heterocyclic compounds containing a thienyl ring, aminopyridine benzimidazole derivatives, triazolyl pyrrolo[2,3-b]pyridine derivatives, N-phenyl imidazolecarboxamides, 7-azaindole derivatives, heteroaryl substituted pyrrolo[2,3-b]pyridine derivatives, 1H-pyrrolo[2,3-b]pyridine derivatives, triazolyl pyrrolo[2,3-b]pyrazine derivatives, N-(3,3,3-trifluoro-2-hydroxo-2-methylpropionyl)-piperidine derivatives, heteroarylcarboxamide derivatives, thiazolecarboxamide derivatives, thiazole-4-carboxamide derivatives, 1,2-dihydroindazolo[4,3-bc][1,5]benzoxazepines, 6-(4-pyrimidinyl)-1H-indazoles, pyrido[4,3-d] pyrimidin-5(6H)-one derivatives, pyridinonyl derivatives, heterocyclic carboxylic acid amides, quinazoline derivatives, pyrimido[5,4-c]quinoline-2,4-diamine derivatives, pyrazole derivatives, benzonaphthyridinone derivatives, 3-aryl-1,2,4-triazole derivatives, resorcinol N-aryl amide derivatives, tetrahydroisoquinoline derivatives, fluorene-amide based derivatives, pyrazole-alcohol derivatives, or combinations thereof.

In some embodiments, the methods for improving exosome production or stimulating exosome secretion by cells include inhibiting lactate dehydrogenase A (LDHA), phosphoglycerate mutase 1 (PGAM1), or monocarboxylate transporter (MCTs). Inhibitors of LDHA, PGAM1, and MCT4 include substituted quinoline derivatives, anthracene-9,10-dione derivatives, or combinations thereof.

In some embodiments, the glycolytic inhibitor comprises a pharmacological agent selected from a halogenated acetate, a monosaccharide or derivative thereof, valerate or derivative thereof, a propionic acid derivative; a pyruvate derivative, or a combination thereof. For example, the glycolytic inhibitor can be selected from 6-fluoro-D-glucose, 2-bromo-D-glucose, 2-fluoro-D-glucose, 2-iodo-D-glucose, glucosyl fluoride, 3-fluoro-D-glucose, 4-fluoro-D-glucose, 1-deoxy-D-glucose, 2-deoxy-D-glucose, 6-deoxy-D-glucose, 6-thio-D-glucose, 5-thio-D-glucose, 6-O-methyl-D-glucose, and valerate, myristate, and palmitate derivatives of 2-dg at 6-O, 4-O, 3-O, and 1-O; 1,1-difluoro-3-phosphate-glycerol, 1,1-difluoro glycerate, 2-fluoro-glyceraldehyde, 2-iodo-glyceraldehyde, 2-thio-glyceraldehyde, 2-methoxy-glyceraldehyde, 2-fluoro-glycerate 2-iodo-glycerate, 2-thio-glycerate, 2-methoxy-glycerate, 3-fluoro-glycerate, 3,3-difluoro-glycerate, enolase 3-iodo-glycerate, 3-carboxylo-glycerate, 3-thio-glycerate, oxamate, 3-halo pyruvate, 2-halo-propionic acid or its salts, 2,2-difluoro-propionic acid, 3-halo-propionic acid, and 2-thiomethyl-acetic acid, mannopyranoses, galactopyranoses, 6-deoxy-6-fluoro-D-glucose, 6-deoxy-6-bromo-D-mannose, 6-deoxy-6-chloro-D-mannose, 6-deoxy-6-fluoro-D-galactose, 6-deoxy-6-chloro-D-galactose, 6-deoxy-6-iodo-D-galactose, 6-deoxy-6-bromo-D-galactose, halogenated C-6 sugars gluconolactones, glucuronic acid, glucopyranoside, and their phosphate derivatives, glucoronides with halogenated glycosides at the C-1 position, C-2 substituted D-hexoses, 2-deoxy-2-halogeno-D-hexoses, 2-deoxy-2-fluoro-D-glucose, 2-chloro-2-deoxy-D-glucose, 2-bromo-D-glucose, 2-iodo-D-glucose, 2-deoxy-2,2-difluoro-D-arabino-hexose, 2-deoxy-2-fluoro-D-mannose, 2-deoxy-D-arabino-hexose, 2-deoxy-2-fluoro-D-galactose, 1,6-anhydro-2-deoxy-2-fluoro-beta-D-glucopyranose, 1-6-anhydrosugar, 2-amino-2-deoxy-D-glucose, glucose amine, 2-amino-2-deoxy D-galactose, galactosamine, 2-amino-2-deoxy-D-mannose, mannosamine, 2-deoxy-2-fluoro-D-mannose, 2-deoxy-2-fluoro-D-galactose, 2-deoxy-D-arabino-hexose, 2-deoxy-2,2-difluoro-D-arabino-hexose, 2-deoxy-2-fluoro-D-glucose 1-Phosphate, 2-deoxy-2-fluoro-D-glucose 6-P, 2-deoxy-2-fluoro-D-glucose 1,6 biphosphate, 2-deoxy-2-fluoro-D-mannose 1-P, 2-deoxy-2-fluoro-D-mannose 6-P, 2-deoxy-2-fluoro-D-mannose 1,6-biphosphate, nucleotide diphosphate, uridine di-P, 1-2 deoxy-2-fluoro-D-glucose, C-2-halogen substituted, and NH₃ substituted derivatives of D-Glucose 6-phosphate, 2-deoxy-2-fluoro-2-D-glucose-6-phosphate, 2-chloro-2-deoxy-D-glucose-6-phosphate, 2-deoxy-D-arabino-hexose-6-phosphate, D-glucosamine-6-phosphate, 2-deoxy-2-fluoro-2-D-manose-6-P, and any known derivatives, C-2 halogenated derivatives of hexose ring pyranoses, mannopyranoses, galactopyranoses, C-2-deoxy-2-fluoropyranoses, and any derivative, C-2 halogenated sugars derivatives, C-2 fluoro-, bromo-, chloro-, or iodo-sugars derivatives, fluoro, bromo, chloro, or iodo C-2 sugars derivatives, gluconolactones, glucuronic acid, glucopyranoside, and their phosphate derivatives, sugars modified at C-1 or C-5 by replacement of hydroxyl by fluorine or deoxygenation or replacement by a sulfur group, glucosyl fluoride, 1-deoxy-D-glucose, 5-thio-D-glucose, 3-deoxy or 3-fluoro-D-glucose or 4-deoxy or 4-fluoro-D-glucose, 2-fluoro- or 2-iodo-, or 2-thio-, or 2-methoxy- or 3-fluoro-, or 3,3 difluoro-, 3-iodo-, or 3-carboxylo-, or 3-thio-glyceraldehydes or glycerates, 3-fluoro-2-phosphoglycerate, phosphothioesters or other phosphor modified analogs, mannoheptulose, mannoheptose, glucoheptose, N-acetylglucosamine, 6-aminonicotinamide acidosis-inducing agents, 2-deoxy-2-fluoro-D-glucose, citrate and halogenated derivatives of citrate, fructose 2,6-bisphosphate, bromoacetylethanolamine phosphate analogues, N-(2-methoxyethyl)˜bromoacetamide, N-(2-ethoxyethyl)-bromoacetamide, N-(3-methoxypropyl)-bromoacetamide), iodoacetate, pentalenolactone, arsenic, 1,1-difluoro-3-phosphate-glycerol, oxamate, 2-fluoro-propionic acid or it salts, 2,2-difluoro-propionic acid, pyruvate modified at C-3 such as 3-halo-pyruvate, 3-halopropionic acid, and 2-thiomethylacetic acid, a stereoisomer, tautomer, racemate, prodrug, metabolite thereof, or a pharmaceutically acceptable salt, base, ester or solvate thereof.

In certain embodiments, the glycolytic inhibitor comprises a thiol reagent. The “thiol reagent” as used herein refers to compounds that are alkylating reagents which modify thiol groups in proteins by S-carboxyamidomethylation or S-carboxymethylation. In some examples, the glycolytic inhibitor comprises a thiol reagents selected from halogenated acetamide or halogenated acetate. Examples of halogenated acetamide and halogenated acetate includes iodoacetamide and iodoacetate, respectively. Preferably, the glycolytic inhibitor comprises iodoacetamide. The essential cysteine residue in the active center of GAPDH can form a thioether bond with iodoacetamide or iodoacetate and can therefore not react anymore with the physiological substrate glyceraldehyde-3-phosphate. As a consequence, GAPDH is inactivated after exposure to iodoacetamide or iodoacetate and glycolysis is inhibited.

In some aspects, methods for improving exosome production or stimulating exosome secretion by cells comprising inhibiting mitochondrial function by contacting the cells with at least one inhibitor of mitochondrial function are disclosed. Mitochondrial function can be inhibited by several mechanisms. For example, mitochondrial function can be inhibited by inhibiting oxidative phosphorylation. Accordingly, methods for improving exosome production or stimulating exosome secretion by cells comprising inhibiting oxidative phosphorylation by contacting the cells with at least one inhibitor of oxidative phosphorylation are disclosed.

Oxidative phosphorylation is associated with a number of enzymes and are known in the art. The enzymes that are considered targets during oxidative phosphorylation include, but are not limited to, enzyme complex I (NADH coenzyme Q reductase), enzyme complex II (succinate-coenzyme Q reductase), enzyme complex III (coenzyme Q cytochrome C reductase), enzyme complex IV (cytochrome oxidase), and enzyme complex V (F0-F1, ATP synthase). It is an aspect of the present disclosure that an inhibitor of mitochondrial function is an inhibitor of an enzyme associated with oxidative phosphorylation. Inhibitors of enzyme complex I are known in the art and can include, but are not limited to any of the following: tritylthioalanine, carminomycin, piperazinedione, rotenone, amytal, 1-methyl-4-phenylpyridinium (MPP+), paraquat, methylene blue, or ferricyanide. Inhibitors of enzyme complex III are known in the art and can include, but are not limited to myxothiazol, antimycin A, ubisemiquinone, cytochrome C, 4,6-diaminotriazine derivatives, methotrexate or electron acceptors such as phenazine methosulfate, and 2,6-dichlorophenol-indophenol. Inhibitors of enzyme complex IV are known in the art and can include, but are not limited to cyanide, hydrogen sulfide, azide, formate, phosphine, carbon monoxide, and ferricyanide. Inhibitors of enzyme complex V are known in the art and can include, but are not limited to VM-26 (4′-demethyl-epipodophyllotoxin thenylidene glucoside), ethylthioalanine, carminomycin, piperazinedione, dinitrophenol, dinitrocresol, 2-hydroxy-3-alkyl-1,4-naphthoquinones, apoptolidin aglycone, oligomycin, ossamycin, cytovaricin, naphthoquinone derivatives (e.g. dichloroallyl-lawsone and lapachol), rhodamine, rhodamine 123, rhodamine QG, carbonyl cyanide p-trifluoromethoxyphenylhydrazone, valinomycin, rothenone, safranine O, cyhexatin, DDT, chlordecone, arsenate, pentachlorophenol, benzonitrile, thiadiazole herbicides, salicylate, cationic amphilic drugs (amiodarone, perhexiline), gramicidin, calcimycin, pentachlorobutadienyl-cysteine (PCBD-cys), trifluorocarbonylcyanide phenylhydrazone (FCCP). Other inhibitors of oxidative phosphorylation can include atractyloside, DDT, free fatty acids, lysophospholipids, n-ethylmaleimide, mersanyl, p-benzoquinone.

In certain embodiments, oxidative phosphorylation can be inhibited by a mitochondrial uncoupler which uncouples phosphorylation from electron transport. In some examples, the methods for improving exosome production or stimulating exosome secretion by cells include inhibiting mitochondrial function by contacting the cells with a mitochondrial uncoupler. Mitochondrial uncoupler can include 2,4-dinitrophenol (DNP), which transfers hydrogen ions from the outer side of the mitochondrion to the matrix and dissipates the proton gradient created by the respiratory chain. Compounds that carry ions across the membrane are called ionophores; DNP acts as a proton ionophore. Other mitochondrial uncoupler includes salicylate.

In other certain embodiments, mitochondrial function can be inhibited by inhibiting the TCA cycle which exists in the matrix of the mitochondria and feeds high energy electrons to the oxidative phosphorylation pathway.

Inhibitors of mitochondrial function, such as by inhibiting oxidative phosphorylation, can be selected from a small molecule, a peptide, a protein, a DNA, a RNA, a siRNA, a miRNA, a shRNA, a large molecule biologic, a polysaccharide, a toxin, or a combination thereof. In some examples, the method for improving exosome production or stimulating exosome secretion by cells can include inhibiting mitochondrial function using a small molecule selected from rhodamine, rotenone, carminomycin, piperazinedione, dinitrocresol, 2-hydroxy-3-alkyl-1,4-naphthoquinones, apoptolidin aglycone, oligomycin, ossamycin, cytovaricin, naphthoquinone derivatives (e.g. dichloroallyl-lawsone and lapachol), rhodamine, rhodamine 123, rhodamine QG, carbonyl cyanide p-trifluoromethoxyphenylhydrazone, valinomycin, rotenone, safranine O, cyhexatin, DDT, chlordecone, arsenate, pentachlorophenol, benzonitrile, thiadiazole herbicides, salicylate, cationic amphilic drugs (amiodarone, perhexiline), gramicidin, calcimycin, pentachlorobutadienyl-cysteine (PCBD-cys), trifluorocarbonylcyanide phenylhydrazone (FCCP), or a combination thereof.

In some embodiments, the methods for improving exosome production or stimulating exosome secretion by cells comprise inhibiting at least one step of the glycolytic pathway by contacting the cells with at least one glycolytic inhibitor, and inhibiting mitochondrial function by contacting the cells with at least one inhibitor of mitochondrial function. Inhibiting the glycolytic pathway and mitochondrial function concurrently is synergistic and significantly stimulates (or increase the production of) exosome secretion.

In some aspects, pharmaceutical compositions comprising one or more inhibitors of mitochondrial function and one or more inhibitors of the glycolytic pathway are disclosed. Such compositions can include one or more inhibitors of oxidative phosphorylation and one or more inhibitors of the enzymes in the glycolytic pathway. An inhibitor of oxidative phosphorylation can include, for example, 2,4-DNP, and an inhibitor of the glycolytic pathway can include iodoacetate. Iodoacetate and 2,4-DNP can be present in a molar ratio from 1:10 to 10:1, from 1:5 to 5:1, from 1:3 to 3:1, from 1:2 to 2:1, preferably in a 1:1 molar ratio.

The pharmaceutical composition can be formulated to primarily inhibit the glycolytic pathway and secondarily inhibit oxidative phosphorylation. In other embodiments, the pharmaceutical composition can be formulated to primarily inhibit oxidative phosphorylation and secondarily inhibit the glycolytic pathway. In further embodiments, the pharmaceutical composition can be formulated to primarily inhibit both oxidative phosphorylation and the glycolytic pathway. Such compositions are formulated according to the dose and efficacy of the inhibitory compounds.

The disclosed methods for improving exosome production or stimulating exosome secretion by cells can be carried out in vitro or in vivo. The exosomes produced in vitro or in vivo can be produced from a cell-of-origin or cell line of interest. The cells of interest for use in the methods disclosed herein can be any cells capable of culture, and can include living, viable cells. The cells can be modified. For example, the cells can be genetically modified to affect a permanent or temporary change in physicochemical features, biodistribution, pharmacokinetics, pharmacodynamics, or biological functions of said extracellular material.

In some embodiments, the methods can use stem cells or progenitor cells. Pluripotent stem cells, adult stem cells, blastocyst-derived stem cells, gonadal ridge-derived stem cells, teratoma-derived stem cells, totipotent stem cells, multipotent stem cells, oncostatin-independent stem cell (OISCs), embryonic stem cells (ES), embryonic germ cells (EG), and embryonic carcinoma cells (EC) are all examples of stem cells. Stem cells can have a variety of different properties and categories of these properties. For example, in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example. Some stem cells can be induced or transformed from non-stem cells by genetic or chemical means.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express Oct4, Sox2, and Nanog. It is understood that some stem cells will express these at the mRNA level, and still others will also express them at the protein level, on for example, the cell surface or within the cell.

In some embodiments, the disclosed methods use a cell other than a stem cell. The adult human body produces many different cell types. These different cell types include, but are not limited to, keratinizing epithelial cells, wet stratified barrier epithelial cells, exocrine secretory epithelial cells, hormone secreting cells, epithelial absorptive cells (gut, exocrine glands and urogenital tract), metabolism and storage cells, barrier function cells (lung, gut, exocrine glands and urogenital tract), epithelial cells lining closed internal body cavities, ciliated cells with propulsive function, extracellular matrix secretion cells, contractile cells, blood and immune system cells, sensory transducer cells, autonomic neuron cells, sense organ and peripheral neuron supporting cells, central nervous system neurons and glial cells, lens cells, pigment cells, germ cells, and nurse cells.

Cells of the human body include keratinizing epithelial cells, epidermal keratinocyte (differentiating epidermal cell), epidermal basal cell (stem cell), keratinocyte of fingernails and toenails, nail bed basal cell (stem cell), medullary hair shaft cell, cortical hair shaft cell, cuticular hair shaft cell, cuticular hair root sheath cell, hair root sheath cell of Huxley's layer, hair root sheath cell of Henle's layer, external hair root sheath cell, hair matrix cell (stem cell), wet stratified barrier epithelial cells, surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, urinary epithelium cell (lining bladder and urinary ducts), exocrine secretory epithelial cells, salivary gland mucous cell (polysaccharide-rich secretion), salivary gland serous cell (glycoprotein enzyme-rich secretion), von Ebner's gland cell in tongue (washes taste buds), mammary gland cell (milk secretion), lacrimal gland cell (tear secretion), ceruminous gland cell in ear (wax secretion), eccrine sweat gland dark cell (glycoprotein secretion), eccrine sweat gland clear cell (small molecule secretion), apocrine sweat gland cell (odoriferous secretion, sex-hormone sensitive), gland of moll cell in eyelid (specialized sweat gland), sebaceous gland cell (lipid-rich sebum secretion), bowman's gland cell in nose (washes olfactory epithelium), Brunner's gland cell in duodenum (enzymes and alkaline mucus), seminal exosome cell (secretes seminal fluid components, including fructose for swimming sperm), prostate gland cell (secretes seminal fluid components), bulbourethral gland cell (mucus secretion), Bartholin's gland cell (vaginal lubricant secretion), gland of Littre cell (mucus secretion), uterus endometrium cell (carbohydrate secretion), isolated goblet cell of respiratory and digestive tracts (mucus secretion), stomach lining mucous cell (mucus secretion), gastric gland zymogenic cell (pepsinogen secretion), gastric gland oxyntic cell (HCl secretion), pancreatic acinar cell (bicarbonate and digestive enzyme secretion), Paneth cell of small intestine (lysozyme secretion), Type II pneumocyte of lung (surfactant secretion), Clara cell of lung, hormone secreting cells, anterior pituitary cell secreting growth hormone, anterior pituitary cell secreting follicle-stimulating hormone, anterior pituitary cell secreting luteinizing hormone, anterior pituitary cell secreting prolactin, anterior pituitary cell secreting adrenocorticotropic hormone, anterior pituitary cell secreting thyroid-stimulating hormone, intermediate pituitary cell secreting melanocyte-stimulating hormone, posterior pituitary cell secreting oxytocin, posterior pituitary cell secreting vasopressin, gut and respiratory tract cell secreting serotonin, gut and respiratory tract cell secreting endorphin, gut and respiratory tract cell secreting somatostatin, gut and respiratory tract cell secreting gastrin, gut and respiratory tract cell secreting secretin, gut and respiratory tract cell secreting cholecystokinin, gut and respiratory tract cell secreting insulin, gut and respiratory tract cell secreting glucagon, gut and respiratory tract cell secreting bombesin, thyroid gland cell secreting thyroid hormone, thyroid gland cell secreting calcitonin, parathyroid gland cell secreting parathyroid hormone, parathyroid gland oxyphil cell, adrenal gland cell secreting epinephrine, adrenal gland cell secreting norepinephrine, adrenal gland cell secreting steroid hormones (mineral corticoids and glucocorticoids), Leydig cell of testes secreting testosterone, theca interna cell of ovarian follicle secreting estrogen, corpus luteum cell of ruptured ovarian follicle secreting progesterone, kidney juxtaglomerular apparatus cell (renin secretion), macula densa cell of kidney, peripolar cell of kidney, mesangial cell of kidney, epithelial absorptive cells (gut, exocrine glands and urogenital tract), intestinal brush border cell (with microvilli), exocrine gland striated duct cell, gall bladder epithelial cell, kidney proximal tubule brush border cell, kidney distal tubule cell, ductulus efferens nonciliated cell, epididymal principal cell, epididymal basal cell, metabolism and storage cells, hepatocyte (liver cell), white fat cell, brown fat cell, liver lipocyte, barrier function cells (lung, gut, exocrine glands and urogenital tract), type i pneumocyte (lining air space of lung), pancreatic duct cell (centroacinar cell), nonstriated duct cell (of sweat gland, salivary gland, mammary gland, etc.), kidney glomerulus parietal cell, kidney glomerulus podocyte, loop of Henle thin segment cell (in kidney), kidney collecting duct cell, duct cell (of seminal exosome, prostate gland, etc.), epithelial cells lining closed internal body cavities, blood vessel and lymphatic vascular endothelial fenestrated cell, blood vessel and lymphatic vascular endothelial continuous cell, blood vessel and lymphatic vascular endothelial splenic cell, synovial cell (lining joint cavities, hyaluronic acid secretion), serosal cell (lining peritoneal, pleural, and pericardial cavities), squamous cell (lining perilymphatic space of ear), squamous cell (lining endolymphatic space of ear), columnar cell of endolymphatic sac with microvilli (lining endolymphatic space of ear), columnar cell of endolymphatic sac without microvilli (lining endolymphatic space of ear), dark cell (lining endolymphatic space of ear), vestibular membrane cell (lining endolymphatic space of ear), stria vascularis basal cell (lining endolymphatic space of ear), stria vascularis marginal cell (lining endolymphatic space of ear), cell of Claudius (lining endolymphatic space of ear), cell of Boettcher (lining endolymphatic space of ear), choroid plexus cell (cerebrospinal fluid secretion), pia-arachnoid squamous cell, pigmented ciliary epithelium cell of eye, nonpigmented ciliary epithelium cell of eye, corneal endothelial cell, ciliated cells with propulsive function, respiratory tract ciliated cell, oviduct ciliated cell (in female), uterine endometrial ciliated cell (in female), rete testis ciliated cell (in male), ductulus efferens ciliated cell (in male), ciliated ependymal cell of central nervous system (lining brain cavities), extracellular matrix secretion cells, ameloblast epithelial cell (tooth enamel secretion), planum semilunatum epithelial cell of vestibular apparatus of ear (proteoglycan secretion), organ of corti interdental epithelial cell (secreting tectorial membrane covering hair cells), loose connective tissue fibroblasts, corneal fibroblasts, tendon fibroblasts, bone marrow reticular tissue fibroblasts, other (nonepithelial) fibroblasts, blood capillary pericyte, nucleus pulposus cell of intervertebral disc, cementoblast/cementocyte (tooth root bonelike cementum secretion), odontoblast/odontocyte (tooth dentin secretion), hyaline cartilage chondrocyte, fibrocartilage chondrocyte, elastic cartilage chondrocyte, osteoblast/osteocyte, osteoprogenitor cell (stem cell of osteoblasts), hyalocyte of vitreous body of eye, stellate cell of perilymphatic space of ear, contractile cells, red skeletal muscle cell (slow), white skeletal muscle cell (fast), intermediate skeletal muscle cell, muscle spindle—nuclear bag cell, muscle spindle—nuclear chain cell, satellite cell (stem cell), ordinary heart muscle cell, nodal heart muscle cell, purkinje fiber cell, smooth muscle cell (various types), myoepithelial cell of iris, myoepithelial cell of exocrine glands, blood and immune system cells, erythrocyte (red blood cell), megakaryocyte, monocyte, connective tissue macrophage (various types), epidermal Langerhans cell, osteoclast (in bone), dendritic cell (in lymphoid tissues), microglial cell (in central nervous system), neutrophil, eosinophil, basophil, mast cell, helper t-lymphocyte cell, suppressor t-lymphocyte cell, killer t-lymphocyte cell, IgM b-lymphocyte cell, IgG b-lymphocyte cell, IgA b-lymphocyte cell, IgE b-lymphocyte cell, killer cell, stem cells and committed progenitors for the blood and immune system (various types), sensory transducer cells, photoreceptor rod cell of eye, photoreceptor blue-sensitive cone cell of eye, photoreceptor green-sensitive cone cell of eye, photoreceptor red-sensitive cone cell of eye, auditory inner hair cell of organ of corti, auditory outer hair cell of organ of corti, Type I hair cell of vestibular apparatus of ear (acceleration and gravity), Type II hair cell of vestibular apparatus of ear (acceleration and gravity), Type I taste bud cell, olfactory neuron, basal cell of olfactory epithelium (stem cell for olfactory neurons), Type I carotid body cell (blood pH sensor), Type II carotid body cell (blood pH sensor), Merkel cell of epidermis (touch sensor), touch-sensitive primary sensory neurons (various types), cold-sensitive primary sensory neurons, heat-sensitive primary sensory neurons, pain-sensitive primary sensory neurons (various types), proprioceptive primary sensory neurons (various types), autonomic neuron cells, cholinergic neural cell (various types), adrenergic neural cell (various types), peptidergic neural cell (various types), sense organ and peripheral neuron supporting cells, inner pillar cell of organ of corti, outer pillar cell of organ of corti, inner phalangeal cell of organ of corti, outer phalangeal cell of organ of corti, border cell of organ of corti, Henson cell of organ of corti, vestibular apparatus supporting cell, Type I taste bud supporting cell, olfactory epithelium supporting cell, Schwann cell, Satellite cell (encapsulating peripheral nerve cell bodies), enteric glial cell, central nervous system neurons and glial cells, neuron cell (large variety of types, still poorly classified), astrocyte glial cell (various types), oligodendrocyte glial cell, lens cells, anterior lens epithelial cell, crystallin-containing lens fiber cell, pigment cells, melanocyte, retinal pigmented epithelial cell, germ cells, oogonium/oocyte, spermatocyte, spermatogonium cell (stem cell for spermatocyte), nurse cells, ovarian follicle cell, Sertoli cell (in testis), and thymus epithelial cell.

In some cases, the cells are mesenchymal stem cells (MSCs) or bone marrow stromal cells (BMSCs). These terms are used synonymously throughout herein. MSCs are of interest because they are easily isolated from a small aspirate of bone marrow, or other mesenchymal stem cell sources, and they readily generate single-cell derived colonies. Bone marrow cells may be obtained from iliac crest, femora, tibiae, spine, rib, knee or other mesenchymal tissues. Other sources of MSCs include embryonic yolk sac, placenta, umbilical cord, skin, fat, synovial tissue from joints, and blood. The presence of MSCs in culture colonies may be verified by specific cell surface markers which are identified with monoclonal antibodies. See U.S. Pat. Nos. 5,486,359 and 7,153,500. The single-cell derived colonies can be expanded through as many as 50 population doublings in about 10 weeks, and can differentiate into osteoblasts, adipocytes, chondrocytes, myocytes, astrocytes, oligodendrocytes, and neurons. In rare instances, the cells can differentiate into cells of all three germlines Thus, MSCs serve as progenitors for multiple mesenchymal cell lineages including bone, cartilage, ligament, tendon, adipose, muscle, cardiac tissue, stroma, dermis, and other connective tissues. See U.S. Pat. Nos. 6,387,369 and 7,101,704. In some cases, MSCs can be defined by a variety of markers. For example, MSCs can be positive for CD73, CD90, CD166 and negative for CD14, CD34 and CD45.

In certain embodiments, the cells can be obtained from a sample or present in a bodily fluid, skin, skeletal muscle, brain, heart, gut, liver, ovarian epithelium, membranous lining of a cavity, umbilical cord, or testis. In some examples, the cells can be obtained from bodily fluid such as from cord blood, peripheral blood, brain, blood vessels, serum, plasma, ascites, urine, cerebrospinal fluid (CSF), sputum, saliva, bone marrow, synovial fluid, aqueous humor, cerumen, broncheoalveolar lavage fluid, semen, prostatic fluid, cowper's fluid or pre-ejaculatory fluid, sweat, fecal matter, tears, cyst fluid, pleural and peritoneal fluid, lymph, chyme, chyle, bile, intestinal fluid, pus, sebum, vomit, mucosal secretion, stool water, pancreatic juice, lavage fluids from sinus cavities, or bronchopulmonary aspirates. In certain embodiments, the cells can be obtained from an organ system. In certain embodiments, the cells can be affected by a disease, such as cancer or any dieses of an organ system. The cells can be obtained from an autologous source or d from an allogeneic source. In certain embodiments, the cells can be obtained from a cell line.

In some embodiments, the cells can be obtained from or are present in a membranous lining in the thoracic cavity, cranial cavity, abdominal cavity, spinal cavity, pelvic cavity, oral cavity, nasal cavity, orbital cavity, bladder, colon, or synovial cavity. The cells of interest can also be first isolated and/or cultured from tissues of interest to increase the number of cells prior to inhibiting.

The cells can be derived from a human or other animal subject. For example, cells can originate from a mouse, guinea pig, rat, cattle, horses, pigs, sheep, or goat. In some embodiments, the cells originate from non-human primates. In some cases, the cells are used as autologous or allogenic treatment.

As described herein, the methods can be used to increase secretion of extracellular vesicles, particularly exosomes. In the methods disclosed herein, the exosomes can be produced in an amount of at least 5 fold, at least 6 fold, at least 7 fold, at least 8 fold, at least 9 fold, at least 10 fold, at least 12 fold, at least 14 fold, at least 15 fold, at least 18 fold, or at least 20 fold compared to uninhibited cells. For example, the cell lines used in the present examples (UMSCC47, PCI-13, Mel526) exhibited an exosome density of from 3×10¹¹ to 8×10¹¹ particles per mL using miniSEC technique. However, the density of the exosomes can vary depending on the cell type used, culture conditions, isolation technique, among other factors.

The exosomes can then be isolated from the cell culture medium. For example, the exosomes can be isolated with size exclusion chromatography, density gradient centrifugation, differential centrifugation, nanomembrane filtration (e.g., ultrafiltration or microfiltration), tangential flow filtration, hydrostatic filtration dialysis, immuno-isolation, affinity purification, microfluidic separation, precipitation, field-flow fractionation, asymmetric flow field-flow fractionation, field-free viscoelastic flow, electrophoretic, acoustic, ion exchange chromatography, fast protein/high performance liquid chromatography (FPLC/HPLC), fluorescence-activated sorting, deterministic lateral displacement (DLD) arrays, or a combination thereof. In some examples, the exosomes can be labeled with a magnetic label, a fluorescent moiety, a radioisotope, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles to aid in isolation or to be reintroduced in vivo as a label for imaging analysis.

Methods of Characterizing a Condition

Methods for characterizing a condition using the exosomes are disclosed herein. The method can include a. stimulating cells in a sample to secrete the exosomes using a method as disclosed herein; b. determining or identifying a biosignature of the exosomes; and c. characterizing the condition based on the biosignature determined or identified. As described herein, stimulating cells to secrete exosomes can be carried out in vitro or in vivo. Accordingly, the methods of characterizing a condition can be carried out in vitro or in vivo.

In some examples, a method for characterizing a condition can comprise stimulating cells in a subject to produce exosomes comprising inhibiting at least one step of the glycolytic pathway by contacting the cells with at least one glycolytic inhibitor, and/or inhibiting mitochondrial function by contacting the cells with at least one inhibitor of mitochondrial function; collecting a sample comprising the exosomes from the subject; determining or identifying a biosignature of the exosomes; and characterizing the condition based on the biosignature. Specifically, the in vivo method can include a) inserting, a device such as a tube into an inlet of a body cavity, b) using the device to contact the cells of interest with a composition to stimulate the cells to produce exosomes, c) collecting (such as by washing the body cavity) a sample comprising the exosomes, d) determining or identifying a biosignature of the exosomes; and e) characterizing the condition based on the biosignature.

Characterizing a condition can be any observable characteristic or trait of cells in a subject, such as a disease or other condition, a disease stage or condition stage, susceptibility to a disease or condition, prognosis of a disease stage or condition, a physiological state, or response to therapeutics. The characteristic can result from a subject's gene expression as well as the influence of environmental factors and the interactions between the two, as well as from epigenetic modifications to nucleic acid sequences. Characterizing a condition can include detecting a disease or condition (including pre-symptomatic early stage detecting), determining the prognosis, diagnosis, or theranosis of a disease or condition, or determining the stage or progression of a disease or condition. Characterizing a condition can also include identifying appropriate treatments or treatment efficacy for specific diseases, conditions, disease stages and condition stages, predictions and likelihood analysis of disease progression, particularly disease recurrence, metastatic spread or disease relapse. A characteristic can also be a clinically distinct type or subtype of a condition or disease, such as a cancer or tumor. In some aspects, the disclosed methods include the analysis of exosomes to provide a biosignature to predict whether a subject is likely to respond to a treatment for a disease or disorder.

In some examples, the methods can be used to determine the presence of or likelihood of developing a tumor, neoplasm, or cancer. The cancer can include a carcinoma, a sarcoma, a lymphoma or leukemia, a germ cell tumor, a blastoma, or other cancers. A cancer detected or assessed by the methods disclosed herein includes, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma.

Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangioendothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma. Lymphoma and leukemia include without limitation chronic lymphocytic leukemia/small lymphocytic lymphoma, B-cell prolymphocytic leukemia, lymphoplasmacytic lymphoma (such as Wald Enstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

The condition can be a premalignant condition, such as actinic keratosis, atrophic gastritis, leukoplakia, erythroplasia, Lymphomatoid Granulomatosis, preleukemia, fibrosis, cervical dysplasia, uterine cervical dysplasia, xeroderma pigmentosum, Barrett's Esophagus, colorectal polyp, or other abnormal tissue growth or lesion that is likely to develop into a malignant tumor. Transformative viral infections such as HIV and HPV also present phenotypes can also be assessed according to the disclosed methods.

In some embodiments, the condition can be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

The condition can also comprise a cardiovascular disease, such as atherosclerosis, congestive heart failure, vulnerable plaque, stroke, or ischemia. The cardiovascular disease or condition can be high blood pressure, stenosis, vessel occlusion or a thrombotic event.

The condition can comprise a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain.

The condition can be an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. Viral proteins, such as HIV or HCV-like particles can be assessed in an exosome, to characterize a viral condition.

The condition can be a perinatal or pregnancy related condition (e.g. preeclampsia or preterm birth), metabolic disease or condition, such as a metabolic disease or condition associated with iron metabolism. For example, hepcidin can be assayed in an exosome to characterize an iron deficiency. The metabolic disease or condition can also be diabetes, inflammation, or a perinatal condition.

The methods disclosed herein can be used to characterize these and other diseases and disorders that can be assessed via biomarkers. Thus, characterizing a condition can be providing a diagnosis, prognosis or theranosis of one of the diseases and disorders disclosed herein.

One or more conditions of a subject can be determined by analyzing one or more exosomes, such as exosomes in a biological sample obtained from the subject. A subject or patient can include, but is not limited to, mammals such as bovine, avian, canine, equine, feline, ovine, porcine, or primate animals (including humans and non-human primates). A subject can also include a mammal of importance due to being endangered, such as a Siberian tiger; or economic importance, such as an animal raised on a farm for consumption by humans, or an animal of social importance to humans, such as an animal kept as a pet or in a zoo. Examples of such animals include, but are not limited to, carnivores such as cats and dogs; swine including pigs, hogs and wild boars; ruminants or ungulates such as cattle, oxen, sheep, giraffes, deer, goats, bison, camels or horses. Also included are birds that are endangered or kept in zoos, as well as fowl and more particularly domesticated fowl, i.e. poultry, such as turkeys and chickens, ducks, geese, guinea fowl. Also included are domesticated swine and horses (including race horses). In addition, any animal species connected to commercial activities are also included such as those animals connected to agriculture and aquaculture and other activities in which disease monitoring, diagnosis, and therapy selection are routine practice in husbandry for economic productivity and/or safety of the food chain.

The subject can have a pre-existing disease or condition, such as cancer. Alternatively, the subject may not have any known pre-existing condition. The subject may also be non-responsive to an existing or past treatment, such as a treatment for cancer.

The biological sample obtained from the subject can be any bodily fluid. For example, the biological sample can be peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation which may be of fetal or maternal origin. The biological sample may also be a tissue sample or biopsy from which exosomes and other circulating biomarkers may be obtained. For example, cells from the sample can be cultured and exosomes isolated from the culture (see Example 1).

In various embodiments, biomarkers or more particularly biosignatures disclosed herein can be assessed directly from such biological samples (e.g., identification of presence or levels of nucleic acid or polypeptide biomarkers or functional fragments thereof) utilizing various methods, such as extraction of nucleic acid molecules from blood, plasma, serum or any of the foregoing biological samples, use of protein or antibody arrays to identify polypeptide (or functional fragment) biomarker(s), as well as other array, sequencing, PCR and proteomic techniques known in the art for identification and assessment of nucleic acid and polypeptide molecules.

The volume of the biological sample used for biomarker analysis can be in the range of between 0.1-20 mL, such as less than about 20, 15, 10, 9, 8, 7, 6, 5, 4, 3, 2, 1 or 0.1 mL.

In some embodiments, exosomes are directly assayed from a biological sample without prior isolation, purification, or concentration from the biological sample. For example, the amount of exosomes in the sample can by itself provide a biosignature that provides a diagnostic, prognostic or theranostic determination. Alternatively, the exosome in the sample may be isolated, captured, purified, or concentrated from a sample prior to analysis. As noted, isolation, capture or purification as used herein comprises partial isolation, partial capture or partial purification apart from other components in the sample. Exosome isolation can be performed using various techniques as described herein, e.g., chromatography, filtration, centrifugation, flow cytometry, affinity capture (e.g., to a planar surface or bead), and/or using microfluidics, as described herein.

An exosome may be purified or concentrated prior to analysis. Analysis of an exosome can include quantitating the amount one or more exosome populations of a biological sample. For example, a heterogeneous population of exosomes can be quantitated, or a homogeneous population of exosomes, such as a population of exosomes with a particular biomarker profile, a particular biosignature, or derived from a particular cell type can be isolated from a heterogeneous population of exosomes and quantitated. Analysis of an exosome can also include detecting, quantitatively or qualitatively, one or more particular biomarker profile or biosignature of an exosome, as described herein.

An exosome can be stored and archived, such as in a bio-fluid bank and retrieved for analysis as necessary. An exosome may also be isolated from a biological sample that has been previously harvested and stored from a living or deceased subject. In addition, an exosome may be isolated from a biological sample which has been collected as described in King et al., Breast Cancer Res 7(5): 198-204 (2005). An exosome can be isolated from an archived or stored sample. Alternatively, an exosome may be isolated from a biological sample and analyzed without storing or archiving of the sample. Furthermore, a third party may obtain or store the biological sample, or obtain or store the exosome for analysis.

An enriched population of exosomes can be obtained from a biological sample. For example, exosomes may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof. Size exclusion chromatography, such as gel permeation columns, centrifugation or density gradient centrifugation, and filtration methods can be used. For example, an exosome can be isolated by differential centrifugation, anion exchange and/or gel permeation chromatography, sucrose density gradients, organelle electrophoresis, magnetic activated cell sorting (MACS), or with a nanomembrane ultrafiltration concentrator. Various combinations of isolation or concentration methods can be used. An exosome can be isolated from a biological sample by filtering a biological sample from a subject through a filtration module and collecting from the filtration module a retentate comprising the exosome, thereby isolating the exosome from the biological sample. The method can comprise filtering a biological sample from a subject through a filtration module comprising a filter; and collecting from the filtration module a retentate comprising the exosome, thereby isolating the exosome from the biological sample. In one embodiment, the filter retains molecules greater than about 100 kiloDaltons. The filtration module can be a component of a microfluidic device.

A binding agent is an agent that binds to a circulating biomarker, such as an exosome or a component of an exosome. The binding agent can be used as a capture agent and/or a detection agent. A capture agent can bind and capture a circulating biomarker, such as by binding a component or biomarker of an exosome. For example, the capture agent can be a capture antibody or capture antigen that binds to an antigen on an exosome. A detection agent can bind to a circulating biomarker thereby facilitating detection of the biomarker. For example, a capture agent comprising an antigen or aptamer that is sequestered to a substrate can be used to capture an exosome in a sample, and a detection agent comprising an antigen or aptamer that carries a label can be used to detect the captured exosome via detection of the detection agent's label. In some embodiments, an exosome is assessed using capture and detection agents that recognize the same exosome biomarkers. For example, an exosome population can be captured using a tetraspanin such as by using an anti-CD9 antibody bound to a substrate, and the captured exosomes can be detected using a fluorescently labeled anti-CD9 antibody to label the captured exosomes. In other embodiments, an exosome is assessed using capture and detection agents that recognize different exosome biomarkers. For example, an exosome population can be captured using a cell-specific marker such as by using an anti-PCSA antibody bound to a substrate, and the captured exosomes can be detected using a fluorescently labeled anti-CD9 antibody to label the captured exosomes. Similarly, the exosome population can be captured using a general exosome marker such as by using an anti-CD9 antibody bound to a substrate, and the captured exosomes can be detected using a fluorescently labeled antibody to a cell-specific or disease specific marker to label the captured exosomes.

A binding agent can be a nucleic acid, protein, or other molecule that can bind to a component of an exosome. The binding agent can comprise DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), dendrimers, or a combination thereof. For example, the binding agent can be a capture antibody. In some embodiments, the binding agent is membrane protein labeling agent. The binding agent can also be a polypeptide or peptide. Polypeptide is used in its broadest sense and may include a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the present invention may be chemically synthesized using standard techniques.

Vesicles such as exosomes can be assessed to provide a characteristic by comparing vesicle characteristics to a reference. In some embodiments, surface antigens on an exosome are assessed. The surface antigens can provide an indication of the anatomical origin and/or cellular origin of the exosomes and other phenotypic information, e.g., tumor status. For example, wherein exosomes found in a patient sample, e.g., a bodily fluid such as blood, serum or plasma, are assessed for surface antigens indicative of colorectal origin and the presence of cancer. The surface antigens may comprise any informative biological entity that can be detected on the exosome membrane surface, including without limitation surface proteins, lipids, carbohydrates, and other membrane components. For example, positive detection of colon derived exosomes expressing tumor antigens can indicate that the patient has colorectal cancer. As such, methods of the invention can be used to characterize any disease or condition associated with an anatomical or cellular origin, by assessing, for example, disease-specific and cell-specific biomarkers of one or more exosomes obtained from a subject.

In another embodiment, one or more exosome payloads are assessed to provide a characteristic. The payload with an exosome comprises any informative biological entity that can be detected as encapsulated within the exosome, including without limitation proteins and nucleic acids, e.g., genomic or cDNA, mRNA, or functional fragments thereof, as well as microRNAs (miRs). In addition, methods disclosed herein are directed to detecting exosome surface antigens (in addition or exclusive to exosome payload) to provide a characterization. For example, exosomes can be characterized by using binding agents (e.g., antibodies or aptamers) that are specific to exosome surface antigens, and the bound exosomes can be further assessed to identify one or more payload components disclosed therein. As described herein, the levels of exosomes with surface antigens of interest or with payload of interest can be compared to a reference to characterize a phenotype. For example, overexpression in a sample of cancer-related surface antigens or exosome payload, e.g., a tumor associated mRNA or microRNA, as compared to a reference, can indicate the presence of cancer in the sample. The biomarkers assessed can be present or absent, increased or reduced based on the selection of the desired target sample and comparison of the target sample to the desired reference sample. Non-limiting examples of target samples include: disease; treated/not-treated; different time points, such as a in a longitudinal study; and non-limiting examples of reference sample: non-disease; normal; different time points; and sensitive or resistant to candidate treatment(s).

Various biomarker molecules can be assessed in biological samples or exosomes obtained from such biological samples. MicroRNAs comprise one class biomarkers assessed via methods disclosed herein. A number of miRNAs are involved in gene regulation, and miRNAs are part of a growing class of non-coding RNAs that is now recognized as a major tier of gene control. Characterization of a number of miRNAs indicates that they influence a variety of processes, including early development, cell proliferation and cell death, apoptosis and fat metabolism. For example, some miRNAs, such as lin-4, let-7, mir-14, mir-23, and bantam, have been shown to play critical roles in cell differentiation and tissue development. Others are believed to have similarly important roles because of their differential spatial and temporal expression patterns. Techniques to isolate and characterize exosomes and miRs are known to those of skill in the art.

A biosignature of the exosomes can be obtained by assessing an exosome population, including surface and payload exosome associated biomarkers, and/or circulating biomarkers including microRNA and protein. A biosignature derived from a subject can be used to characterize a phenotype of the subject. A biosignature can further include the level of one or more additional biomarkers, e.g., circulating biomarkers or biomarkers associated with an exosome of interest. A biosignature of an exosome of interest can include particular antigens or biomarkers that are present on the exosome. The biosignature can also include one or more antigens or biomarkers that are carried as payload within the exosome, including the microRNA under examination. The biosignature can comprise a combination of one or more antigens or biomarkers that are present on the exosome with one or more biomarkers that are detected in the exosome. The biosignature can further comprise other information about an exosome aside from its biomarkers. Such information can include exosome size, circulating half-life, metabolic half-life, and specific activity in vivo or in vitro. The biosignature can comprise the biomarkers or other characteristics used to build a classifier.

A characteristic of an exosome in and of itself can be assessed to determine a biosignature. The characteristic can be used to diagnose, detect or determine a disease stage or progression, the therapeutic implications of a disease or condition, or characterize a physiological state. Such characteristics include without limitation the level or amount of exosomes, exosome size, temporal evaluation of the variation in exosome half-life, circulating exosome half-life, metabolic half-life of an exosome, or activity of an exosome.

Biomarkers that can be included in a biosignature include one or more proteins or peptides (e.g., providing a protein signature), nucleic acids (e.g. RNA signature as described, or a DNA signature), lipids (e.g. lipid signature), or combinations thereof. In some embodiments, the biosignature can also comprise the type or amount of drug or drug metabolite present in an exosome, (e.g., providing a drug signature), as such drug may be taken by a subject from which the biological sample is obtained, resulting in an exosome carrying the drug or metabolites of the drug.

A biosignature can also include an expression level, presence, absence, mutation, variant, copy number variation, truncation, duplication, modification, or molecular association of one or more biomarkers. A genetic variant, or nucleotide variant, refers to changes or alterations to a gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant may occur in transcriptional regulatory regions, untranslated regions of mRNA, exons, introns, or exon/intron junctions. The genetic variant may or may not result in stop codons, frame shifts, deletions of amino acids, altered gene transcript splice forms or altered amino acid sequence.

In an embodiment, nucleic acid biomarkers, including nucleic acid payload within an exosome, is assessed for nucleotide variants. The nucleic acid biomarker may comprise one or more RNA species, e.g., mRNA, miRNA, snoRNA, snRNA, rRNAs, tRNAs, siRNA, hnRNA, shRNA, or a combination thereof. Similarly, DNA payload can be assessed to form a DNA signature. An RNA signature or DNA signature can also include a mutational, epigenetic modification, or genetic variant analysis of the RNA or DNA present in the exosome. Epigenetic modifications include patterns of DNA methylation. See, e.g., Lesche R. and Eckhardt F., DNA methylation markers: a versatile diagnostic tool for routine clinical use. Curr Opin Mol Ther. 2007 June; 9(3):222-30, which is incorporated herein by reference in its entirety. Thus, a biomarker can be the methylation status of a segment of DNA.

A biosignature used to characterize a condition can comprise one or more biomarkers. The biomarker can be a circulating marker, a membrane associated marker, or a component present within an exosome or on an exosome's surface. These biomarkers include without limitation a nucleic acid (e.g. RNA (mRNA, miRNA, etc.) or DNA), protein, peptide, polypeptide, antigen, lipid, carbohydrate, or proteoglycan.

The biosignature can include the presence or absence, expression level, mutational state, genetic variant state, or any modification (such as epigenetic modification, post-translation modification) of a biomarker. The expression level of a biomarker can be compared to a control or reference, to determine the overexpression or under expression (or upregulation or downregulation) of a biomarker in a sample. In some embodiments, the control or reference level comprises the amount of a same biomarker, such as a miRNA, in a control sample from a subject that does not have or exhibit the condition or disease. In another embodiment, the control of reference levels comprises that of a housekeeping marker whose level is minimally affected, if at all, in different biological settings such as diseased versus non-diseased states. In yet another embodiment, the control or reference level comprises that of the level of the same marker in the same subject but in a sample taken at a different time point. Other types of controls are described herein.

Nucleic acid biomarkers include various RNA or DNA species. For example, the biomarker can be mRNA, microRNA (miRNA), small nucleolar RNAs (snoRNA), small nuclear RNAs (snRNA), ribosomal RNAs (rRNA), heterogeneous nuclear RNA (hnRNA), ribosomal RNAS (rRNA), siRNA, transfer RNAs (tRNA), or shRNA. The DNA can be double-stranded DNA, single stranded DNA, complementary DNA, or noncoding DNA. miRNAs are short ribonucleic acid (RNA) molecules which average about 22 nucleotides long. miRNAs act as post-transcriptional regulators that bind to complementary sequences in the three prime untranslated regions (3′ UTRs) of target messenger RNA transcripts (mRNAs), which can result in gene silencing. One miRNA may act upon 1000s of mRNAs. miRNAs play multiple roles in negative regulation, e.g., transcript degradation and sequestering, translational suppression, and may also have a role in positive regulation, e.g., transcriptional and translational activation. By affecting gene regulation, miRNAs can influence many biologic processes. Different sets of expressed miRNAs are found in different cell types and tissues.

Biomarkers for use with the invention further include peptides, polypeptides, or proteins, which terms are used interchangeably throughout unless otherwise noted. In some embodiments, the protein biomarker comprises its modification state, truncations, mutations, expression level (such as overexpression or under expression as compared to a reference level), and/or post-translational modifications, such as described above. In a non-limiting example, a biosignature for a disease can include a protein having a certain post-translational modification that is more prevalent in a sample associated with the disease than without.

A biosignature may include a number of the same type of biomarkers (e.g., two different microRNA species) or one or more of different types of biomarkers (e.g. mRNAs, miRNAs, proteins, peptides, ligands, and antigens).

A biosignature can be detected qualitatively or quantitatively by detecting a presence, level or concentration of a microRNA, exosome or other biomarkers, as disclosed herein. These biosignature components can be detected using a number of techniques known to those of skill in the art. For example, a biomarker can be detected by microarray analysis, polymerase chain reaction (PCR) (including PCR-based methods such as real time polymerase chain reaction (RT-PCR), quantitative real time polymerase chain reaction (Q-PCR/qPCR) and the like), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or combinations thereof. A biomarker, such as a nucleic acid, can be amplified prior to detection. A biomarker can also be detected by immunoassay, immunoblot, immunoprecipitation, enzyme-linked immunosorbent assay (ELISA; EIA), radioimmunoassay (RIA), flow cytometry, or electron microscopy (EM).

Biosignatures can be detected using capture agents and detection agents, as described herein. A capture agent can comprise an antibody, aptamer or other entity which recognizes a biomarker and can be used for capturing the biomarker. Biomarkers that can be captured include circulating biomarkers, e.g., a protein, nucleic acid, lipid or biological complex in solution in a bodily fluid. Similarly, the capture agent can be used for capturing an exosome. A detection agent can comprise an antibody or other entity which recognizes a biomarker and can be used for detecting the biomarker exosome, or which recognizes an exosome and is useful for detecting an exosome. In some embodiments, the detection agent is labeled and the label is detected, thereby detecting the biomarker or exosome. The detection agent can be a binding agent, e.g., an antibody or aptamer. In other embodiments, the detection agent comprises a small molecule such as a membrane protein labeling agent. See, e.g., the membrane protein labeling agents disclosed in Alroy et al., US. Patent Publication US 2005/0158708. In an embodiment, exosomes are isolated or captured as described herein, and one or more membrane protein labeling agent is used to detect the exosomes. In many cases, the antigen or other exosome-moiety that is recognized by the capture and detection agents are interchangeable. As a non-limiting example, consider an exosome having a cell-of-origin specific antigen on its surface and a cancer-specific antigen on its surface. In one instance, the exosome can be captured using an antibody to the cell-of-origin specific antigen, e.g., by tethering the capture antibody to a substrate, and then the exosome is detected using an antibody to the cancer-specific antigen, e.g., by labeling the detection antibody with a fluorescent dye and detecting the fluorescent radiation emitted by the dye. In another instance, the exosome can be captured using an antibody to the cancer specific antigen, e.g., by tethering the capture antibody to a substrate, and then the exosome is detected using an antibody to the cell-of-origin specific antigen, e.g., by labeling the detection antibody with a fluorescent dye and detecting the fluorescent radiation emitted by the dye.

In some embodiments, a same biomarker is recognized by both a capture agent and a detection agent. This scheme can be used depending on the setting. In one embodiment, the biomarker is sufficient to detect an exosome of interest, e.g., to capture cell-of-origin specific exosomes. In other embodiments, the biomarker is multifunctional, e.g., having both cell-of-origin specific and cancer specific properties. The biomarker can be used in concert with other biomarkers for capture and detection as well.

The methods provided herein can be used in identifying a novel biosignature of an exosome, such as one or more novel biomarkers for the diagnosis, prognosis or theranosis of a phenotype. In one embodiment, one or more exosomes can be isolated from a subject with a phenotype and a biosignature of the one or more exosomes determined. The biosignature can be compared to a subject without the phenotype. Differences between the two biosignatures can be determined and used to form a novel biosignature. The novel biosignature can then be used for identifying another subject as having the phenotype or not having the phenotype.

Differences between the biosignature from a subject with a particular condition can be compared to the biosignature from a subject without the particular condition. The one or more differences can be a difference in any characteristic of the exosome. For example, the level or amount of exosomes in the sample, the half-life of the exosome, the circulating half-life of the exosome, the metabolic half-life of the exosome, or the activity of the exosome, or any combination thereof, can differ between the biosignature from the subject with a particular condition and the biosignature from the subject without the particular condition.

In some embodiments, one or more biomarkers differ between the biosignature from the subject with a particular condition and the biosignature from the subject without the particular condition. For example, the expression level, presence, absence, mutation, variant, copy number variation, truncation, duplication, modification, molecular association of one or more biomarkers, or any combination thereof, may differ between the biosignature from the subject with a particular phenotype and the biosignature from the subject without the particular condition. The biomarker can be any biomarker disclosed herein or that can be used to characterize a biological entity, including a circulating biomarker, such as protein or microRNA, an exosome, or a component present in an exosome or on the exosome, such as any nucleic acid (e.g. RNA or DNA), protein, peptide, polypeptide, antigen, lipid, carbohydrate, or proteoglycan.

Method of Treating a Condition

Also disclosed herein is a method of treating a subject in need, the method comprising administering to the subject the exosome released by the cells, as disclosed herein. The methods described herein can be used to treat a variety of diseases and disorders, including, but not limited to cancer, diseases of an organ system, graft-versus-host disease, pulmonary hypertension, arthritis, ocular trauma, kidney failure, among others. The compositions disclosed herein can be used to promote angiogenesis, wound healing and skin regeneration. For example, the compositions disclosed herein can be disposed on a patch, such as a hydrogel patch.

The exosome disclosed herein can comprise cargo for delivery to a subject in need thereof. The cargo can be conjugated to exosome, embedded within exosome, encapsulated within exosome, or otherwise carried by exosome. Alternatively, the exosome itself can be the cargo. Thus, as used herein, a reference to a cargo being “present” in exosome is understood to include any of the foregoing means of carrying the cargo.

In some embodiments, the exosome is loaded with 2-5 molecules or copies of a single cargo or two (or more) different cargos. In some embodiments, an exosome or pharmaceutical composition thereof is loaded with 1-4,000, 10-4,000, 50-3,500, 100-3,000, 200-2,500, 300-1,500, 500-1,200, 750-1,000, 1-2,000, 1-1,000, 1-500, 10-400, 50-300, 1-250, 1-100, 2-50, 2-25, 2-15, 2-10, 3-50, 3-25, 3-25, 3-10, 4-50, 4-25, 4-15, 4-10, 5-50, 5-25, 5-15, or 5-10 molecules or copies of a single cargo or two (or more) different cargos. The cargo is endogenous or exogenous, and where two or more cargos are present each cargo is independently endogenous or exogenous.

A cargo can be an endogenous cargo, an exogenous cargo, or a combination thereof. Examples of cargos that can be conjugated, embedded, encapsulated within or otherwise carried by an exosome described herein include, without limitation, nucleic acid molecules (e.g., DNA, cDNA, antisense oligonucleotides, mRNA, inhibitory RNAs (e.g., anti sense RNAs, miRNAs, small interfering RNAs (siRNAs), short hairpin RNAs (shRNAs), and agomiRs), antagomiRs, primary miRNAs (pri-miRNAs), long non-coding RNAs (lncRNAs), small nuclear RNA (snRNA), small nucleolar RNA (snoRNA), and microbial RNAs), polypeptides (e.g., enzymes, antibodies), lipids, hormones, vitamins, minerals, small molecules, and pharmaceuticals, or any combination thereof.

Exosome as described herein, can include one or more cargos, wherein the cargo(s) is a therapeutic molecule. Exemplary small molecules include, without limitation, chemotherapeutic agent, antibiotics, steroids, sterols, peptides, natural products, alkaloids, terpenes, and synthetic molecules.

The cargo can be either diagnostic or therapeutic in nature. When used for diagnostic purposes, the cargo can comprise, for example, an imaging agent. As used herein, an imaging agent is an agent that emits signal directly or indirectly thereby allowing its detection Imaging agents such as contrast agents and radioactive agents that can be detected using medical imaging techniques such as nuclear medicine scans and magnetic resonance imaging (MRI) are disclosed herein. Also disclosed are imaging agents for fluorescence imaging such as fluorescent dyes or dye-conjugated nanoparticles. In other embodiments, the agent to be delivered is conjugated, or fused to, or mixed or combined with an imaging agent.

A therapeutic molecule can be conjugated to an exosome, embedded within an exosome, encapsulated within an exosome, or otherwise carried by an exosome or any combination thereof. Examples of therapeutic agents include, without limitation, peptide, protein, DNA, RNA, siRNA, miRNA, shRNA, small molecule, large molecule biologic, polysaccharide, lipid, toxin, mRNAs and/or polypeptides encoded by the mRNAs (e.g., Cre recombinase, insulin, peptide hormones, and enzymes), miRNAs, siRNAs, or miRNA antagonists of therapeutic value, nutrients that may be unstable or have low bioavailability (e.g., vitamins B 1 and B 12, polyunsaturated fatty acids), pharmaceuticals (e.g., antibiotics (such as puromycin, gentamycin, and neomycin), cancer drugs (such as chemotherapeutics, immunotherapies, hormone therapies, and targeted therapies), activators of Toll-like receptors), and molecules to be delivered to macrophages (e.g., to remove or prevent atherosclerotic plaques, or treat macrophage-related cancers), as well as any of the other therapeutic cargo molecules provided herein.

In some embodiments, the therapeutic agent is a biologic. In some embodiments, the biologic is selected from a hormone, allergen, adjuvant, antigen, immunogen, vaccine, interferon, interleukin, growth factor, monoclonal antibody (mAb). In some embodiments, the biologic is a polypeptide, or a peptide.

In some embodiments, the exosome can be engineered to partially or completely deplete all or select elements of the cargo, to change the desired payload specifically or in preparation of additional means of cargo loading. Methods of engineering exosome, such as exosomes, are known in the prior art and are described in Dhruvitkumar et al. (2017) Low active loading of cargo into engineered extracellular exosomes results in inefficient miRNA mimic delivery, Journal of Extracellular Exosomes, 6:1, herein incorporated by reference in its entirety for its teaching concerning extracellular exosome and cargo.

By way of non-limiting illustration, examples of certain embodiments of the present disclosure are given below.

### EXAMPLES

### Example 1: Stimulation of Exosome Secretion by Sodium Iodoacetate and 2,4-dinitrophenol

Abstract: Exosome secretion by cells is a complex, poorly understood process. Studies of exosomes would be facilitated by a method for increasing their production and release. Here, we present a method for stimulating the secretion of exosomes. Cultured cells were treated or not with sodium iodoacetate (IAA; glycolysis inhibitor) plus 2,4-dinitrophenol (DNP; oxidative phosphorylation inhibitor). Exosomes were isolated by size-exclusion chromatography and their morphology, size, concentration, cargo components and functional activity were compared. IAA/DNP treatment (up to 10 μM each) was non-toxic and resulted in a 3 to 16-fold increase in exosome secretion. Exosomes from IAA/DNP-treated or untreated cells had similar biological properties and functional effects on endothelial cells (SVEC4-10). IAA/DNP increased exosome secretion from mouse organ cultures, and in vivo injections enhanced the levels of circulating exosomes. IAA/DNP decreased ATP levels (p<0.05) in cells. A cell membrane-permeable form of 2′,3′-cAMP and 3′-AMP mimicked the potentiating effects of IAA/DNP on exosome secretion. In cells lacking 2′,3′-cyclic nucleotide 3′-phosphodiesterase (CNPase; an enzyme that metabolizes 2′,3′-cAMP into 2′-AMP), effects of IAA/DNP on exosome secretion were enhanced. The IAA/DNP combination is a powerful stimulator of exosome secretion, and these stimulatory effects are, in part, mediated by intracellular 2′,3′-cAMP.

Introduction: Cells growing in culture were treated with sodium iodoacetate (IAA) and 2,4-dinitrophenol (DNP), and intracellular ATP, ADP and AMP were extracted and quantified by HPLC. Toxicity was evaluated by LDH assays. Exosomes were isolated from treated and untreated cells by mini size exclusion chromatography (mini-SEC) and characterized by electron microscopy, tunable resistive pulse sensing and immunoblotting. Functional activity of exosomes was measured in co-cultures with SVEC4-10 lympho-endothelial cells. Mouse kidneys and livers were harvested and cultured in the presence or absence of IAA/DNP, and exosomes were isolated from the conditioned media. IAA/DNP was injected daily into C57BL/6J mice for 14 days and exosomes in the plasma were isolated and evaluated on days 0, 7 and 14. Organs from treated mice were cultured and exosome release was compared to organs obtained from control mice.

Treatment with IAA/DNP resulted in an intracellular decrease of ATP and an increase of AMP levels in a dose dependent manner IAA/DNP effects were found to be reversible and caused no toxic effects at concentrations up to 10 μM. Exosome secretion was stimulated in a concentration-dependent manner for different cell types (HPV+head and neck cancer cells UMSCC47: 16-fold increase; HPV(−) head and neck cancer cells PCI-13: 24-fold increase; metastatic melanoma cells, Mel526: 3-fold increase; lympho-endothelial cells SVEC4-10: 7-fold increase). Exosomes from treated or untreated cells showed a similar morphology, size and marker expression profiles. No differences in the internalization by SVEC4-10 cells or the stimulation of SVEC4-10 migration were observed for exosomes derived from treated or untreated cells. Exosome secretion from organ cultures was stimulated depending on the dose of IAA/DNP. In vivo injections of IAA/DNP enhanced the levels of circulating exosomes in the blood of mice.

The data show that the combination of IAA/DNP is a potent stimulator of exosome secretion in vitro and in vivo. The present disclosure adds new options to the culture techniques for generating exosomes that might be used in drug-delivery or therapeutic applications in nanomedicine. Further, the disclosure will significantly contribute to a better understanding of exosome-mediated effects and their clinical significance as disease biomarkers or as drug delivery vehicles.

Materials and Methods:

Cell lines: Cells lines included in this example are listed in Table 1. All cell lines were grown at 37° C. in the atmosphere of 5% CO₂ in air. Cultures were supplemented with fetal bovine serum (FBS) depleted of exosomes by ultracentrifugation at 100,000×g for 3 h. Cells were cultured in 150 cm² cell culture flasks using 25 ml of culture medium. Media used for cell cultures are described in Table 1. Seeding protocol was optimized for each cell type as recently described in Ludwig N., et al., (Exp Cell Res. 2019; 378(2):149-57). After seeding, cells were allowed to attach to the flask for 6 h, were then treated with indicated reagents and incubated for 48 or 72 h as indicated.

TABLE 1
Cell lines included in this study^{a}

| Cell lines | Cell type | Origin | Source | Media |
|---|---|---|---|---|
| UMSCC47 | HPV(+) head | Dr. Thomas | Robert L. Ferris | DMEM (Lonza Inc.), |
| | and neck cancer | Carey | (UPMC Hillman | 1% (v/v) |
| | cells | (University of | Cancer Center) | penicillin/streptomycin, |
| | | Michigan) | | 10% (v/v) exosome- |
| | | | | depleted FBS (Gibco, |
| | | | | Thermo Fisher |
| | | | | Scientific) |
| PCI-13 | HPV(−) head | Established and | | DMEM, 1% (v/v) |
| | and neck cancer | maintained in | | penicillin/streptomycin, |
| | cells | inventors | | 10% (v/v) exosome- |
| | | laboratory | | depleted FBS |
| Mel526 | Metastatic | Marincola et al. | Walter J. Storkus | RPMI-1640, 1% (v/v) |
| | melanoma cells | | (Department of | penicillin/streptomycin, |
| | | | Immunology, | 10% (v/v) exosome- |
| | | | University of | depleted FBS |
| | | | Pittsburgh) | |
| SVEC4-10 | Endothelial | O'Connell and | ATCC, Manassas, | DMEM, 1% (v/v) |
| | cells | Edidin | VA, USA, cat. # | penicillin/streptomycin, |
| | | | CRL-2181 | 10% (v/v) exosome- |
| | | | | depleted FBS |

^{a}Cell lines included in this study and information about their type, origin and source as well as media used for their culture.

Exosome isolation by mini-SEC: Processing of supernatants and exosome isolation by mini-SEC was performed as previously described in Ludwig et al. Briefly, cell culture supernatants were centrifuged at room temperature (RT) for 10 min at 2000×g, were transferred to new tubes for centrifugation at 10,000×g at 4° C. for 30 min and filtrated using a 0.22 μm bacterial filter. Afterwards, aliquots of supernatants were concentrated by using Vivacell 100 concentrators at 2000×g. 1 mL of concentrated supernatant was loaded on a 10 cm-long Sepharose 2-B column and individual 1 mL fractions were collected. Fraction #4 containing non-aggregated exosomes was used in subsequent assays. The established isolation technique fulfils the criteria of the MISEV2018 guidelines and therefore the term ‘exosomes’ is used.

Protein concentration: Protein concentrations were determined by using a BCA protein assay (Pierce Biotechnology, Rockford, Ill., USA) according to the manufacturer's instructions.

Transmission electron microscopy (TEM): TEM was performed as previously described in Ludwig et al. Freshly isolated TEX were placed on copper grids coated with 0.125% Formvar in chloroform and stained with 1% (v/v) uranyl acetate in ddH2O. A JEM 1011 microscope was used for TEX visualization.

Tunable resistive pulse sensing (TRPS): Size distribution and concentrations of the particles in isolated exosome fractions were analyzed using tunable-resistive pulse sensing (TRPS) by qNano (Izon) as described in Ludwig N, et al., Mol Cancer Res. 2018; 16(11):1798-808.

Western blot analysis: To concentrate isolated exosomes, 0.5 mL 100K Amicon Ultra centrifugal filters (EMD Millipore) were used for centrifugation at 4000×g. Each lane was loaded with 5 μg of fraction #4 proteins, and PVDF membranes were incubated overnight at 4° C. with a TSG101 antibody (1:1000, ab30871, Abcam, Cambridge, Mass.) as previously described in Ludwig N, et al.

Cell migration: Cell migration by SVEC4-10 endothelial cells was analyzed as previously described in Ludwig N, et al. Briefly, 5×10⁴ SVEC4-10 cells were starved in serum-free media overnight and were added to the upper compartment of 24-well transwell plates with 8 μm pore diameter (Corning). Cells migrated towards serum-free medium or the medium supplemented with 10 μg exosomes derived from UMSCC47 cells treated with 0, 1 or 10 μM of DNP/IAA or 10% FBS, which were added to the lower compartment. After 6 h of incubation at 37° C., non-migrating cells in the upper chamber were removed with cotton swabs. Migrating cells on the lower surface of the membrane were fixed in methanol and stained with 0.2% crystal violet (Sigma-Aldrich). The number of migrated cells was counted in a light microscope in six randomly selected regions of interest at 20× magnification using an Olympus BX51 microscope (Olympus America, Center Valley, Pa.).

Uptake of exosomes by SVEC4-10 cells: 5×10³ SVEC4-10 cells were seeded in 24-well plates and incubated for 24 h. 5 or 10 μg of TEX isolated from UMSCC47 cells treated with 0, 1 or 10 μM of DNP/IAA were labeled with SYTO® RNASelect™ Green Fluorescent Cell Stain (Invitrogen) using manufacturer's instructions and added to the cell culture for 4 h. Cells were washed twice with PBS and were harvested. Mean fluorescence intensity (MFI) of the cells was determined using a flow cytometer (Gallios Flow Cytometer; Beckman Coulter, Miami, Fla., USA). Data were analyzed using Kaluza software (version 1.0; Beckman Coulter).

Isolation of exosomes from tissue explants: Kidneys were harvested from 6 week old female C57BL/6 mice in an aseptic manner and immediately cultured in 6-well plates using 5 ml of DMEM supplemented with 1% (v/v) penicillin/streptomycin for 48 h as described by Mincheva-Nilsson et al. (Curr Protoc Immunol. 2016; 2016 (November):14.42.1-14.42.21). Tissue explants were treated with indicated concentrations of IAA/DNP. The treatment was given to intact tissue explants, minced tissue or was injected with an insulin syringe (29 G×½″, Exelint, Redondo Beach, Calif., USA) at three different locations. Supernatant was collected by gentle aspiration including washing of the tissue and the walls of the culture vessel. Processing of supernatants and exosome isolations were performed as described above.

LDH assay: LDH release of cultured cells was performed using Pierce LDH Cytotoxicity Assay Kit (Thermo Scientific) following the manufacturer's instructions. Cells were cultured for 72 h with indicated concentrations of IAA/DNP.

Extraction and quantitation of NAD+, ATP, ADP, and AMP: ATP, ADP, and AMP were measured in cells treated with 0, 1 and 10 μM IAA/DNP using HPLC. The detailed protocol for the extraction of NAD+, ATP, ADP and AMP was previously described in Long K R, et al. (Mol Biol Cell. 2017; mbc.E17-04-0211).

Isolation of rat CNPASE +/+ and −/− PGVSMCs: 2′,3′-Cyclic nucleotide 3′-phosphodiesterase (CNPase) knockout rats used in this example were generated by the MCW Gene Editing Rat Resource Program. This strain was produced by injecting a CRISPR targeting the sequence GCTACTGCCGCCGGGACATC into rat embryos. The resulting mutation was a 7 base pair deletion in exon 2. Animals were genotyped by PCR. Preglomerular vascular smooth muscle cells (PGVSMCs) were isolated from kidneys of wild type (CNPase +/+) and knockout (CNPase −/−) rats using a previously described method. Cells were cultured at 37° C. in the atmosphere of 5% CO₂ in air using DMEM supplemented with exosome-depleted FBS. Exosome isolation was performed as described above.

Animal study: This study was carried out in strict accordance with the recommendations in the Guide for the Care and Use of Laboratory Animals of the NIH. The protocol (18042580) was approved by the institutional Animal Care and Use Committee of the University of Pittsburgh (Animal Welfare Assurance Number: D16-00118). Female C57BL/6 mice aged 6 to 8 weeks were purchased from Jackson Laboratories. All animal experiments were conducted according to the protocols approved by the Institutional Animal Care and Use Committee. IAA/DNP was daily injected intraperitoneal for 14 days in a concentration of 0.195 or 0.975 μmoles in volume of 100 PBS injections of the same volume served as vehicle control. Blood was collected by submandibular bleeding on the days 0, 7 and 14 of the experiment. Plasma was isolated by centrifuging at 1000×g for 10 min and further processed by spinning 30 min at 10,000×g and filtered by a 0.22 μm filter. Next, exosomes were isolated as described above. Additionally, kidneys and livers were harvested from all animals at day 14 and were cultured for 48 hours. The supernatant of organ cultures was collected and exosomes were isolated as described above.

Statistical analysis: All data were analysed using the GraphPad Prism software (v7.0). Values are expressed as means±SD or SEM as indicated in the figure legends. Differences between groups were assessed by Student t test and differences were considered significant at p<0.05.

Results

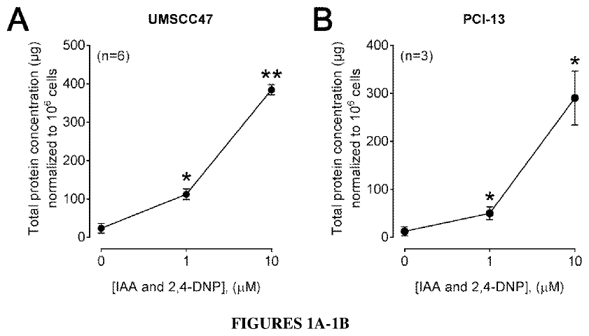

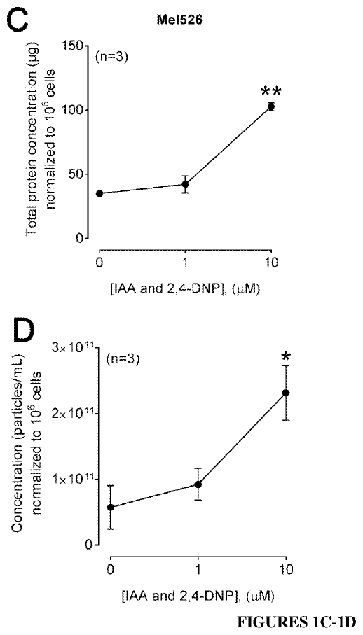

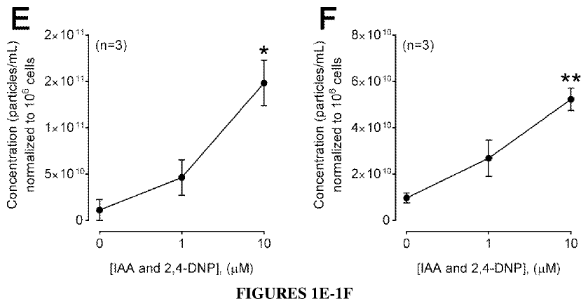

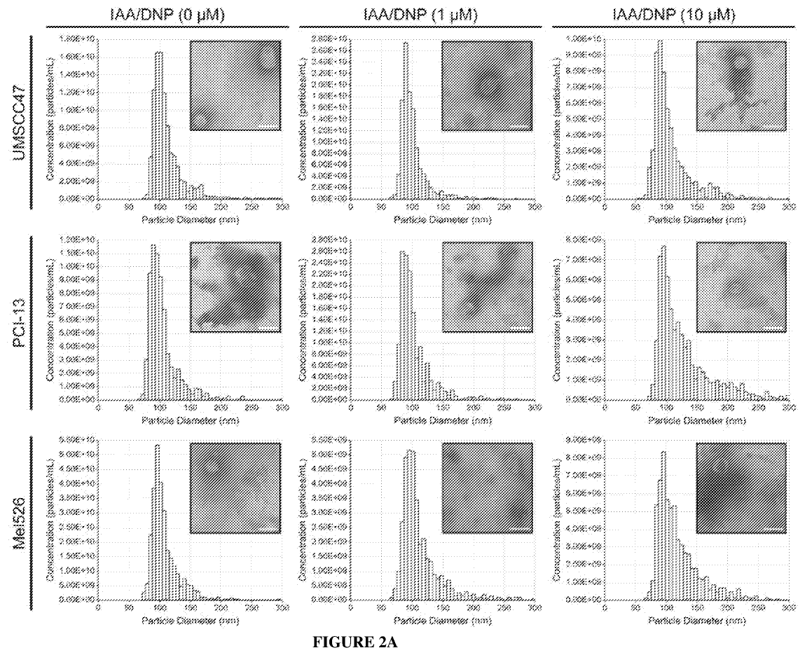

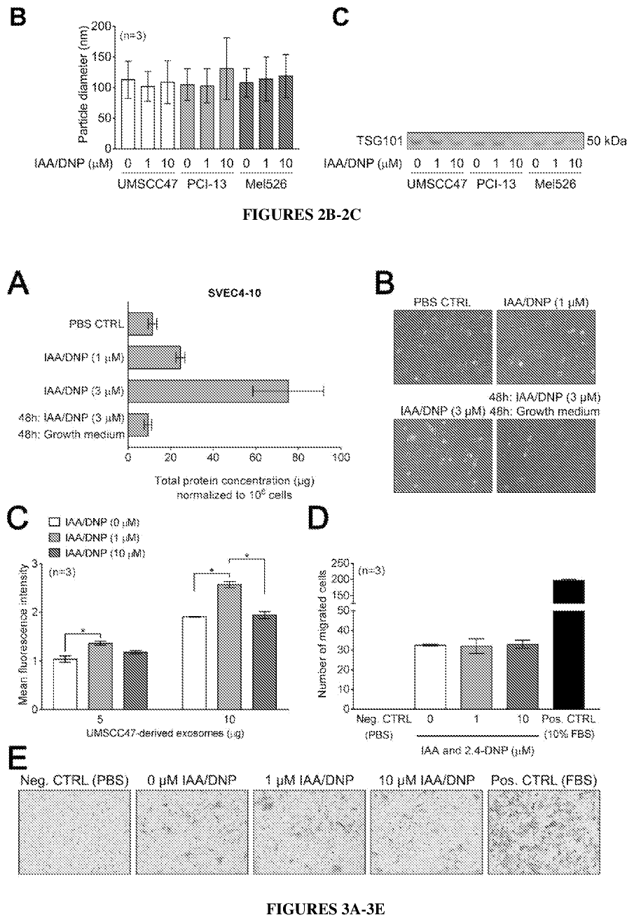

IAA/DNP stimulates exosome secretion in vitro: Exosomes were isolated from cancer cell lines UMSCC47, PCI-13 and MEL526, which were treated with 0, 1 or 10 μM IAA/DNP for 72 h. All cell cultures showed a concentration-dependent increase of exosomes in the conditioned medium quantified by BCA protein assays and expressed in μg as total exosomal protein as shown in FIG. 1A-1C. Treatments of UMSCC47 cells with 10 μM IAA/DNP revealed a 3-fold increase of exosome secretion after 6 h, an almost 6-fold increase after 12 h and ≥10-fold increase for cellular treatments longer 48 h. Results were validated by qNano, which measures particle size and concentrations (FIG. 1D-1F). The particle size in fraction #4 of all three cell lines measured by qNano ranged from approximately 60 to 160 nm, and no alterations in exosome size were observed comparing exosomes derived from treated or untreated cells (FIGS. 2A and 2B). Exosomes isolated from cells treated with increasing concentrations of IAA/DNP showed similar morphology by TEM (FIG. 2A). All exosomes carried TSG101, which indicated their origin from the endocytic compartment of the parent cell (FIG. 2C).

SVEC4-10 lympho-endothelial cells were used to confirm effects of IAA/DNP on normal (non-malignant) cells. IAA/DNP increased exosome secretion by SVEC4-10 in a concentration-dependent manner (FIG. 3A).

To measure biological activity of exosomes derived from treated or untreated cells, functional studies with SVEC4-10 lympho-endothelial cells were performed. Exosomes were internalized by SVEC4-10 cells within 4 h, and the same concentration of exosomes derived from treated or untreated cells had similar functional activity. SVEC4-10 cells internalized slightly more exosomes derived from cells treated with 1 μM IAA/DNP (FIG. 3C). The migration of SVEC4-10 cells was similarly stimulated by exosomes from treated or untreated cells (FIGS. 3D and 3E).

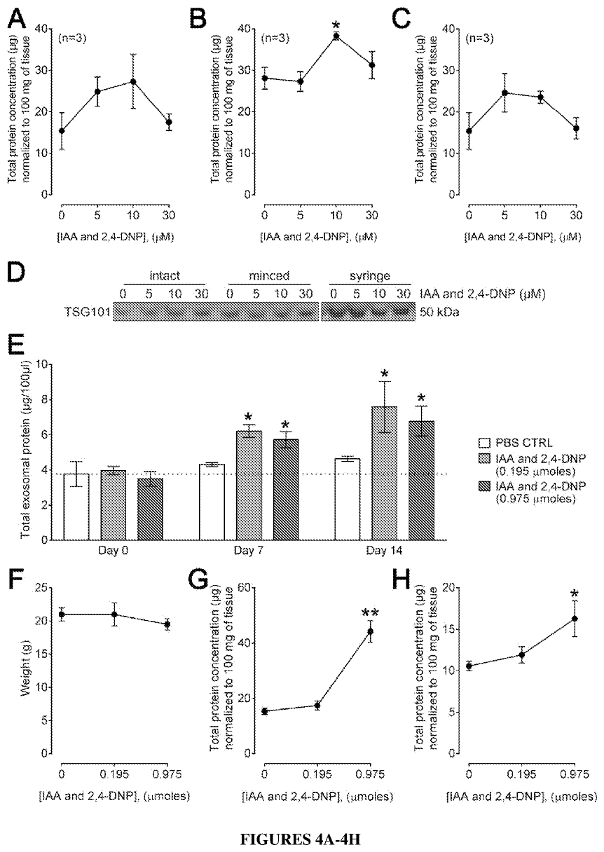

IAA/DNP combination stimulates exosome secretion ex vivo: To investigate further the stimulatory effects of IAA/DNP on exosome secretion, tissue explants (kidneys) were harvested from C57BL/6 mice and cultured for 48 h in the presence or absence of IAA/DNP. Some kidneys were minced and other kidneys were left intact. Intact kidneys also received injections of IAA/DNP at three sites using a syringe. IAA/DNP caused a concentration-dependent increase of exosome release from tissue explants into culture medium. The concentration of 10 μM IAA/DNP was found to be most effective for the intact and minced tissues, whereas the tissue explants treated with IAA/DNP injections already responded to 5 μM IAA/DNP (FIGS. 4A, 4B and 4C) Similar TSG101 levels were detected in all exosome samples regardless of the concentration of IAA/DNP used (FIG. 4D).

IAA/DNP stimulates exosome secretion in vivo: In vitro and ex vivo results were validated by injecting IAA/DNP into mice. Based on the amount of body fluid of mice, a dose of 0.195 μmoles of IAA/DNP was used to provide an initial concentration of 10 μM in the body fluids. Another group of mice received a 5-fold higher dose (0.975 μmoles). The injections did not affect the weight of the animals and did not alter their behaviour or induced signs of stress or pain (FIG. 4F). Both doses of IAA/DNP stimulated the levels of circulating exosomes in the blood compared to control mice (FIG. 4E). Kidneys and livers of mice were harvested and cultured for 48 h after 14 days of treatment with IAA/DNP. Notably, exosome levels were elevated in both tissue types in a dose-dependent manner (FIGS. 4G and 4H).

IAA/DNP causes a non-toxic energy depletion in cultured cells: The numbers of dead cells in the culture medium measured indirectly by LDH assays showed low levels of LDH in the culture medium up to concentrations of 10 μM IAA/DNP. However, 15 μM IAA/DNP led to a very slight, but statistically significant (p=0.033), increase in LDH. Therefore, 10 μM was used as the highest concentration of IAA/DNP in subsequent assays and was considered as a non-toxic dose.

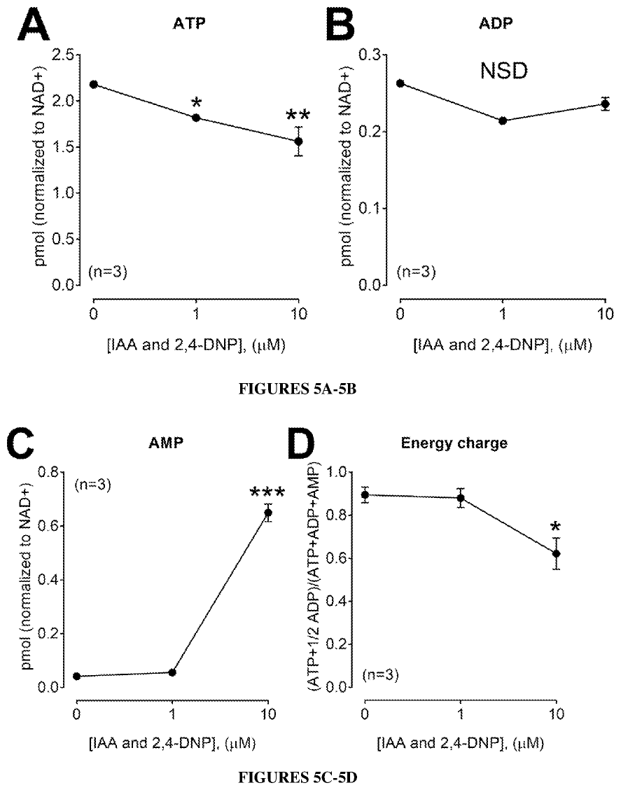

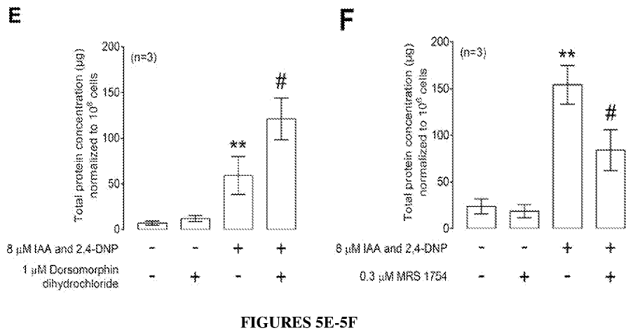

To further characterize the effects of IAA/DNP, HPLC was used to quantify levels of ATP, ADP and AMP after treatment of cells with 0, 1 and 10 μM IAA/DNP. Data were normalized to account for differences in cell number between conditions. In cultured cells, IAA/DNP decreased ATP levels (FIG. 5A) but increased AMP levels (FIG. 5C), and these effects were concentration dependent. ADP levels were not affected by IAA/DNP treatment (FIG. 5B). Calculating the energy status of the cells using the formula (ATP+1/2 ADP)/(ATP+ADP+AMP) revealed a significant drop of the energy charge (FIG. 5D).

To test the toxicity of IAA/DNP, SVEC4-10 were cultured in the presence of IAA/DNP for 48 h followed by 48 h of culture in the regular growth medium. This led to exosome levels which were comparable to those in untreated cells, indicating that the treatment with IAA/DNP is reversible and non-toxic (FIGS. 3A and 3B).

Stimulation of exosome secretion by IAA/DNP is augmented by AMPK inhibition and attenuated by A_{2B}R antagonism: Stimulation of exosome production by IAA/DNP was significantly augmented by an inhibitor (dorsomorphin) of AMP-activated protein kinase (AMPK) (p<0.05, FIG. 5E). In contrast, stimulation of exosome production by IAA/DNP was significantly decreased by the A_{2B}R antagonist MRS 1754 (p<0.05, FIG. 5F).

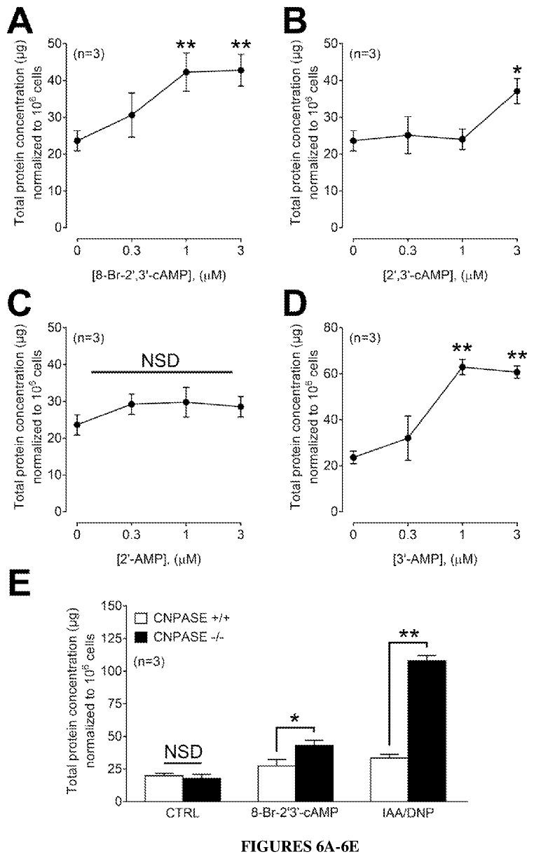

8-Br-2′,3′-cAMP enhances exosome production, and the effects of both 8-Br-2′,3′-cAMP and IAA/DNP are augmented in CNPase knockout cells: 2′,3′-cAMP (not to be confused with the 2^{nd} messenger 3′,5′-cAMP) is a recently described endogenous non-canonical cyclic nucleotide, the production of which is stimulated by energy depletion with IAA/DNP in a concentration-dependent manner Extracellular 2′,3′-cAMP is metabolized to 2′-AMP and 3′-AMP, which in turn are metabolized to adenosine. Importantly, intracellular 2′,3′-cAMP opens mitochondrial permeability transition pores (mPTPs) and triggers stress granule formation. Thus extracellular 2′,3′-cAMP can engage A_{2B}R via adenosine, and intracellular 2′,3′-cAMP can compromise cellular energy production via opening mPTPs and stimulating the production of stress granules. Therefore, it is conceivable that the effects of IAA/DNP on exosome production are mediated in part by 2′,3′-cAMP. To test this, the effects of a cell membrane permeable form of 2′,3′-cAMP were examined, namely 8-Br-2′,3′-cAMP, on exosome production. As shown in FIG. 6A, 8-Br-2′,3′-cAMP led to a concentration-dependent increase in exosome secretion (p<0.01). Exogenous 2′,3′-cAMP, which has very limited cell membrane permeability, also stimulated exosome production, but only at high concentrations (p<0.05, FIG. 6B). Because extracellular 2′,3′-cAMP is metabolized to extracellular 2′-AMP and/or 3′-AMP, which in turn are converted to extracellular adenosine, the effects of both exogenous 2′-AMP and 3′-AMP on exosome secretion were also examined Although 2′-AMP did not affect exosome secretion (FIG. 6C), 3′-AMP significantly stimulated exosome release (p<0.01, FIG. 6D). Because CNPase metabolizes intracellular 2′,3′-cAMP, the release of exosomes induced by either 8-Br-2′,3′-cAMP or IAA/DNP in PGVSMCs obtained from CNPase +/+versus CNPase −/− rats were examined. Under basal conditions, exosome release was similar in CNPase +/+versus CNPase −/− cells (FIG. 6E). In contrast, stimulation of exosome production in PGVSMCs by either 8-Br-2′,3′-cAMP or IAA/DNP was greater in CNPase −/− compared with CNPase +/+ cells (FIG. 6E; *p<0.05 and **p<0.01, respectively).

Discussion

The clinical use of exosomes both as biomarkers of disease (e.g., cancer, critical illness or cardiovascular diseases) and carriers of drugs and biologics is of great current interest. One of the most crucial limitations to achieve clinical use is the purification of exosomes in sufficiently large quantities. Although isolation techniques are constantly improving and several methods have been suggested to stimulate exosome release, the reported techniques only yield limited quantities of exosomes and indicate that the need for an exosome stimulant remains unmet.

It has been reported that the combination of DNP and IAA induces a potent reduction of cellular energy charge by simultaneously blocking oxidative phosphorylation and glycolysis. It was reasoned that blocking cellular pathways of energy production using DNP and IAA might be an effective strategy for releasing exosomes. Indeed, as shown in this example, IAA/DNP is a powerful stimulator of exosome secretion in cultured cells and in animal models.

It is believed that IAA/DNP is the most effective method yet discovered to stimulate exosome release, and in the present disclosure, it is more efficacious than other commonly used stimulators of exosome secretion. Datta et al. screened the effects of 4580 pharmacologically compounds on exosome release and only 6 were found to be activators of exosome biogenesis with forskolin being the most potent one (6-fold increase). Also, IAA/DNP is safe, can be used both in vitro and in vivo and works across a variety of cell lines. It can therefore accelerate exosome research and be used as a tool for the generation of exosomes in different settings. The protein-based quantification of circulating exosomes in a complex biofluid, such as mouse plasma, may also detect co-isolated non-exosome associated proteins. In particular, LDL, VLDL and chylomicron contaminations have been reported and might contribute to the heterogeneity of exosomes isolated from plasma.

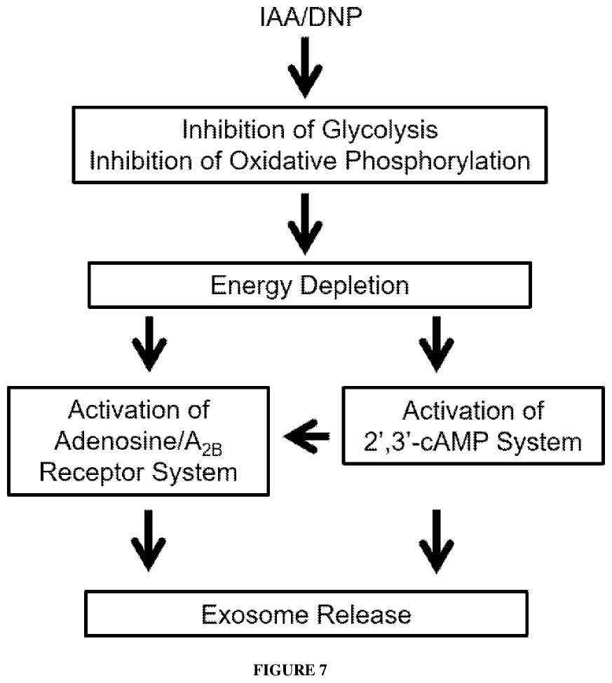

The underlying mechanisms for the elevated exosome secretion after IAA/DNP treatment are summarized in FIG. 7. IAA inhibits glycolysis and DNP inhibits oxidative phosphorylation and, thereby, the combination severely suppresses energy charge. Decreased ATP and increased AMP levels in IAA/DNP-treated cells confirm energy depletion. Further, AMP accumulation triggers two processes: 1) activation of adenosine receptors (ARs) via adenosine production from AMP, and 2) activation of AMPK. The present example shows that blocking A_{2B}Rs attenuates the effects of IAA/DNP and blocking of AMPK augments IAA/DNP effects on exosome release. Therefore, it was conclude that, in part, the ability of IAA/DNP to increase exosome release is mediated via A_{2B}Rs; but likely activation of AMPK attenuates the effects of IAA/DNP. Because IAA/DNP combination increases 2′,3′-cAMP, the role of endogenous 2′,3′-cAMP in exosome release by using 8-Br-2′,3′-cAMP and by using CNPase knockout cells. These experiments confirmed that 2′,3′-cAMP plays a role in the IAA/DNP-mediated release of exosomes from cells. Although the mechanism by which 2′,3′-cAMP increases exosome release remains unknown, it could involve: 1) formation of adenosine; 2) inhibition of mitochondrial function by opening mPTPs; 3) formation of stress granules, which would block protein synthesis; and 4) other “direct” effects of 2′,3′-cAMP on the process of exosome secretion. Besides activating the adenosine pathway and the 2′,3′-cAMP axis there may be other effects triggered by energy depletion induced by IAA/DNP.

IAA/DNP is believed to be the most effective method yet discovered to stimulate exosome release that involves, at least, A_{2B}Rs and 2′,3′-cAMP. This method allows for a harvest of ample exosomes from various cells and may serve as a platform technology for the development of exosome-based therapies in the future.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1-4. (canceled)

5. A method for improving exosome production or stimulating exosome secretion by cells, the method comprising:
a) inhibiting at least one step of the glycolytic pathway by contacting the cells with at least one glycolytic inhibitor, and
b) inhibiting mitochondrial function by contacting the cells with at least one inhibitor of mitochondrial function.

6. (canceled)

7. The method of claim 5, wherein the glycolytic inhibitor is a pharmacological agent that inhibits glyceraldehyde-3-phosphate dehydrogenase.

8. The method of claim 5, wherein the glycolytic inhibitor is a pharmacological agent selected from a halogenated acetate, a monosaccharide or derivative thereof, valerate or derivative thereof, a propionic acid derivative, a pyruvate derivative, or a combination thereof.

9. The method of claim 5, wherein the glycolytic inhibitor is a halogenated acetate.

10. The method of claim 5, wherein the inhibitor of mitochondrial function is a pharmacological agent that inhibits oxidative phosphorylation.

11. The method of claim 5, wherein the inhibitor of mitochondrial function is 2,4-dinitrophenol (DNP).

12. The method of claim 5, wherein the method includes contacting the cells with iodoacetate and 2,4-dinitrophenol.

13. The method of claim 12, wherein iodoacetate and 2,4-dinitrophenol are present in a molar ratio from 1:10 to 10:1.

14. The method of claim 5, wherein the method is carried out in vitro.

15. The method of claim 14, wherein the cells are obtained from a sample of bodily fluid, skin, skeletal muscle, brain, heart, gut, liver, ovarian epithelium, membranous lining of a cavity, umbilical cord, or testis.

16. (canceled)

17. (canceled)

18. The method of claim 5, wherein the method is carried out in vivo.

19. The method of claim 18, wherein the cells are present in a tissue, a membranous lining, or stem cells in a subject.

20. (canceled)

21. The method of claim 5, wherein the exosomes are produced in an amount of at least 5 fold compared to uninhibited cells.

22. A method for characterizing a condition comprising:
a. stimulating cells in a sample to produce exosomes comprising inhibiting at least one step of the glycolytic pathway by contacting the cells with at least one glycolytic inhibitor, and/or inhibiting mitochondrial function by contacting the cells with at least one inhibitor of mitochondrial function;
b. determining or identifying a biosignature of the isolated exosomes; and
c. characterizing the condition based on the biosignature.

23. (canceled)

24. The method of claim 22, wherein step a, stimulating cells in a sample is carried out in vitro.

25. The method of claim 22, wherein step a, stimulating cells in a sample, is carried out in vivo in a subject.

26. The method of claim 25, further comprising collecting a sample comprising the exosomes from the subject after stimulation and prior to determining or identifying the biosignature of the exosomes.

27. (canceled)

28. (canceled)

29. The method of claim 22, wherein the glycolytic inhibitor is a halogenated acetate, wherein the inhibitor of mitochondrial function is 2,4-dinitrophenol (DNP), or a combination thereof.

30-39. (canceled)

40. A method of treating a condition in a subject, comprising administering to the subject a composition comprising exosomes prepared by a method according to claim 5.

41-47. (canceled)

48. A composition comprising:
exosomes, wherein the exosomes are prepared by a method according to claim 5, wherein the exosomes are present in an amount of at least 5 fold, compared to uninhibited cells,
at least one glycolytic inhibitor, and/or
at least one inhibitor of mitochondrial function.
